(19) United States
(12) Patent Application Publication
(10) Pub. No.: US 2025/0073292 A1
(43) Pub. Date: Mar. 6, 2025

(54) PELARGONIUM GRAVEOLENS EXTRACT FOR THE TREATMENT OF TRAUMATIC BRAIN INJURIES

(71) Applicants: THE STATE OF ISRAEL, MINISTRY OF AGRICULTURE & RURAL DEVELOPMENT, AGRICULTURAL R, Rishon Lezion (IL); SHEBA IMPACT LTD., Ramat Gan (IL)

(72) Inventors: Sigal LIRAZ ZALTSMAN, Bnei-Atarot (IL); Anat ELMANN, Rehovot (IL); Nativ DUDAI, Kfar Yehezkel (IL)

(73) Assignees: THE STATE OF ISRAEL, MINISTRY OF AGRICULTURE & RURAL DEVELOPMENT, AGRICULTURAL R, Rishon Lezion (IL); SHEBA IMPACT LTD., Ramat Gan (IL)

(21) Appl. No.: 18/293,996

(22) PCT Filed: Aug. 2, 2022
(86) PCT No.: PCT/IL2022/050836

§ 371 Date: Jan. 31, 2024

Related U.S. Application Data
(60) Provisional application No. 63/203,840, filed on Aug. 2, 2021.

(51) Int. Cl. A61K 36/185; A61K 31/045; A61P 25/00; A61P 25/28

(52) U.S. Cl. CPC A61K 36/185; A61K 31/045; A61P 25/00; A61P 25/28; A61K 2236/331

(57) ABSTRACT

The present invention provides methods and compositions comprising essential oil extracted from Pelargonium graveolens for treating, preventing, or ameliorating traumatic brain injury (TBI) in a subject, or for improving cognitive functions in a subject suffering from TBI.

### TECHNOLOGICAL FIELD

The present disclosure generally relates to a novel treatment for brain damage.

### BACKGROUND ART

References considered to be relevant as background to the presently disclosed subject matter are listed below:
1. Lifshitz J. Experimental CNS Trauma: A General Overview of Neurotrauma Research. In: Kobeissy F H, ed. Brain Neurotrauma: Molecular, Neuropsychological, and Rehabilitation Aspects. Boca Raton (FL) 2015.
2. Maas, A. I., et al., Collaborative European NeuroTrauma Effectiveness Research in Traumatic Brain Injury (CENTER-TBI): a prospective longitudinal observational study. Neurosurgery, 2015. 76(1): p. 67-80.
3. Maas, A. I. R., et al., Traumatic brain injury: integrated approaches to improve prevention, clinical care, and research. Lancet Neurol, 2017. 16(12): p. 987-1048.
4. Gardner R C, Burke J F, Nettiksimmons J, Kaup A, Barnes D E, Yaffe K. Dementia risk after traumatic brain injury vs nonbrain trauma: the role of age and severity. JAMA neurology. December 2014; 71(12):1490-1497.
5. Nordstrom P, Michaelsson K, Gustafson Y, Nordstrom A. Traumatic brain injury and young onset dementia: a nationwide cohort study. Annals of neurology. March 2014; 75(3):374-381.
6. Beauchamp K, Mutlak H, Smith W R, Shohami E, Stahel P F. Pharmacology of traumatic brain injury: where is the “golden bullet”? Molecular medicine. November-December 2008; 14(11-12):731-74.0.
7. Narayan R K, Michel M E, Ansell B, et al. Clinical trials in head injury. Journal of Neurotrauma. May 2002; 19(5):503-557.
8. Abbasloo E, Dehghan F, Khaksari M, et al. The anti-inflammatory properties of Satureja khuzistanica Jamzad essential oil attenuate the effects of traumatic brain injuries in rats. Scientific reports. Aug. 18, 2016; 6:31866.
9. Elmann, A., Mordechay, S., Rindner, M., and Ravid, U. (2010). Anti-neuroinflammatory effects of the essential oil from Pelargonium graveolens in microglial cells. Journal of Functional foods. 2: 17-22.
10. WO 2013/168090
11. Ravid U. and Putievsky E. (1984). The influence of harvest dates and leaf location on the essential oil content and major components of Pelargonium Graveolens L. Acta Horticulturae 144:159-165.
12. Putievsky E, Ravid U and Dudai N (1990). The effect of water stress on yield components and essential oil of Pelargonium Graveolens L. J. Ess. Oil Res., 2:111-114.
13. Chen Y, Constantini S, Trembovler V, Weinstock M, Shohami E. An experimental model of closed head injury in mice: pathophysiology, histopathology, and cognitive deficits. Journal of neurotrauma. October 1996; 13(10):557-568.
14. Flierl M A, Stahel P F, Beauchamp K M, Morgan S J, Smith W R, Shohami E. Mouse closed head injury model induced by a weight-drop device. Nature protocols. 2009; 4(9):1328-1337.
15. Beni-Adani, L., Gozes, I., Cohen, Y., Assaf, Y., Steingart, R. A., Brenneman, D. E., Eizenberg, O., Trembolver, V., Shohami, E., 2001. A peptide derived from activity-dependent neuroprotective protein (ADNP) ameliorates injury response in closed head injury in mice. J Pharmacol Exp Ther 296, 57-63.
16. Tsenter J, Beni-Adani L, Assaf Y, Alexandrovich A G, Trembovler V, Shohami E., 2008. Dynamic changes in the recovery after traumatic brain injury in mice: effect of injury severity on T2-weighted MRI abnormalities, and motor and cognitive functions. J Neurotrauma 25(4):324-33.
17. Fox G. B., Fan L., LeVasseur R. A., Faden A. I, 1998. Effect of traumatic brain injury on mouse spatial and nonspatial learning in the Barnes circular maze. J Neurotrauma 15(12):1037-46.
18. Pitts M. W., 2018 Barnes Maze Procedure for Spatial Learning and Memory in Mice. Bio Protoc. 8(5): e2744.
19. Young K. and Morrison H., 2018 Quantifying Microglia Morphology from Photomicrographs of Immunohistochemistry Prepared Tissue Using ImageJ J Vis Exp. 136: 57648.

Acknowledgement of the above references herein is not to be inferred as meaning that these are in any way relevant to the patentability of the presently disclosed subject matter.

### BACKGROUND

Traumatic brain injury (TBI) affects millions of people each year, worldwide and is a major cause of death and disability among young people [1]. The World Health Organization has declared TBI a significant public health problem, and the US Department of Defense (DoD) has reported that more than 313,816 service personnel suffer brain damage [2, 3]. A traumatic head injury causes immediate symptoms as well as chronic symptoms that may last for many years after the injury among them, cognitive decline manifested in decreased ability to learn, remember, and concentrate, and the onset of dementia at a relatively young age [4, 5].

There is currently no effective pharmacological treatment for both immediate and long-term effects and cognitive decline. These consequences harm the quality of life of the victim, his family and constitute a great economic burden on the health system, and the caring family [6, 7].

Abbasloo et al [8] disclose that intraperitoneal injection of Satureja khuzistanica Jamzad essential oil attenuates the effects of traumatic brain injuries in rats.

The essential oil extracted from the plant Pelargonium graveolens was previously shown to inhibit activation of microglial cells in culture [9]. WO 2013/168090 [10] discloses that a Pelargonium graveolens constituent (i.e. (S) (−) citronellol) inhibits in vitro the activity of the enzyme acetyl choline esterase. In addition, Pelargonium graveolens oil (also termed “geranium oil”) was shown to be effective in ameliorating various physiological parameters of Parkinson's disease in an animal model of the disease (MPTP).

There is an urgent need to develop a treatment that can slow down or eliminate the late onset symptoms and the cognitive decline associated with TBI and thereby to reduce the vast personal, social, and economic burden of these debilitating conditions.

### GENERAL DESCRIPTION

In one aspect, the present invention provides a pharmaceutical or nutritional composition for treating, preventing, or ameliorating traumatic brain injury in a subject, the composition comprising essential oil extracted from Pelargonium graveolens and one or more physiologically acceptable carriers.

In another aspect, the present invention provides a pharmaceutical or nutritional composition for improving cognitive function of a subject suffering from traumatic brain injury, the composition comprising essential oil extracted from Pelargonium graveolens and one or more physiologically acceptable carriers.

In another one of its aspects, the present invention provides Pelargonium graveolens essential oil extract for use in a method of treating, preventing, or ameliorating TBI in a subject in need thereof, the method comprising administering to the subject a therapeutically effective amount of said Pelargonium graveolens essential oil extract.

In another one of its aspects, the present invention provides Pelargonium graveolens essential oil extract for use in a method of improving cognitive function of a subject suffering from traumatic brain injury, the method comprising administering to the subject a therapeutically effective amount of said Pelargonium graveolens essential oil extract.

In yet another one of its aspects, the present invention provides a method of treating, preventing, or ameliorating TBI, the method comprising administering to a subject in need thereof a therapeutically effective amount of Pelargonium graveolens essential oil extract or a composition comprising Pelargonium graveolens essential oil extract.

In yet another one of its aspects, the present invention provides a method of improving cognitive function of a subject suffering from traumatic brain injury, the method comprising administering to said subject a therapeutically effective amount of Pelargonium graveolens essential oil extract or a composition comprising Pelargonium graveolens essential oil extract.

In some embodiments, said cognitive function is at least one of a learning ability, memory (e.g., spatial memory) or anxiety.

In one embodiment, the TBI is caused by an external force, rapid acceleration, blast waves, or penetration of the skull that reaches brain tissue.

In one embodiment, said subject is a human.

In one embodiment, said subject is a mammal selected from the group consisting of sheep, pigs, cattle, goats, horses, camels, buffalo, rabbits, cats, dogs, and primates.

In one embodiment, said composition is in the form of a food article, a beverage, a food additive, food supplement, botanical drug, or a pharmaceutical composition.

In one embodiment, said composition is in the form of tablets, capsules, liquid syrups, nasal spray, nasal drops, soft gels, suppositories, patches, and enemas.

In one embodiment, said composition is a pharmaceutical composition comprising a pharmaceutically acceptable carrier.

In one embodiment, said pharmaceutical composition is administered by oral, intraperitoneal, subcutaneous, transcutaneous, topical, intramuscular, intraarticular, subconjunctival, intranasal, or intraocular administration.

In one embodiment, said composition is administered at a concentration of between about 1 mg/kg and 50 mg/kg.

In one embodiment, said composition is administered once, twice, three times or more daily, or once every two days or every three days for a treatment session of between about 5 days and four weeks, or more following the TBI, or administered in a prophylactic manner to subjects at risk of TBI.

In one embodiment the composition is administered chronically.

In one embodiment, said composition is administered in a treatment session that comprises between about 8 and about 12 daily treatments, e.g., 9 daily treatments per session.

In one embodiment, said composition is administered in a treatment session that comprises between about 8 and about 12 daily treatments, e.g., 9 daily treatments per session, followed by chronic administration once a week.

In one embodiment, at least one additional treatment session with said composition is administered to said subject between about 4 months, 5 months, 6 months, 7 months, 8 months, 9 months, 10 months, 11 months, 12 months, or more following the TBI.

In one embodiment, said composition is provided orally at a concentration of between about 5 mg/kg and about 50 mg/kg once a day or once every two or three days for a treatment session that comprises between about 8 and about 12 daily treatments per session, e.g., 9 daily treatments per session.

In one embodiment, said composition is provided by intranasal administration at a concentration of between about 1 mg/kg and about 5 mg/kg once a day or once every two or three days for a treatment session that comprises between about 8 and about 12 daily treatments per session, e.g., 9 daily treatments per session.

In one embodiment, said composition is provided by inhalation at a concentration of between about 0.1% and about 10% once a day or once every two or three days for a treatment session that comprises between about 8 and about 12 daily treatments per session, e.g., 9 daily treatments per session.

In one embodiment, one additional treatment session of two weeks is provided to said subject 9 months following the TBI.

In one embodiment, the composition is a sustained release composition.

In another aspect, the present invention provides a pharmaceutical or nutritional composition for treating, preventing, or ameliorating traumatic brain injury in a subject, the composition comprising isolated Citronellol and one or more physiologically acceptable carriers.

In another aspect, the present invention provides a pharmaceutical or nutritional composition for improving cognitive function of a subject suffering from traumatic brain injury, the composition comprising isolated citronellol and one or more physiologically acceptable carriers.

In another aspect, the present invention provides isolated Citronellol for use in a method of treating, preventing, or ameliorating TBI in a subject in need thereof, the method comprising administering to the subject a therapeutically effective amount of said isolated Citronellol.

In another aspect, the present invention provides isolated citronellol for use in a method of improving cognitive function of a subject suffering from traumatic brain injury, the method comprising administering to the subject a therapeutically effective amount of said isolated citronellol.

In another aspect, the present invention provides a method of treating, preventing, or ameliorating TBI, the method comprising administering to a subject in need thereof a therapeutically effective amount of isolated Citronellol.

In another aspect, the present invention provides a method of improving cognitive function of a subject suffering from traumatic brain injury, the method comprising administering to said subject a therapeutically effective amount of isolated citronellol.

In certain embodiments, said isolated Citronellol is administered at a concentration of between about 1 mg/kg and 15 mg/kg, e.g., 4 mg/kg or 10 mg/kg.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIG. 1 is a graph showing Neurological severity score (NSS) in the following groups of mice: mice which were traumatized and treated with the vehicle (CHI+vehicle), mice which were traumatized and treated with 200 mg/kg Pg oil (CHI+200 mg/kg Pg), and mice which were traumatized and treated with 500 mg/kg Pg oil (CHI+500 mg/kg Pg). NSS was measured at several time points following injury. Mice were fed with Pg oil 24 hours after the trauma for 2 weeks, with a total of 9 doses. CHI+vehicle: n=39 rounds-7, CHI+500 mg/kg: n=30 rounds-5, and CHI+200 mg/kg: n=28 rounds-4. Results are presented as mean±SEM. [*] indicates a P-value of <0.05, [**] indicates a P-value of <0.01, [***] indicates a P-value of <0.001 and [****] indicates a P-value of <0.0001. The P-values refer to the comparison of the groups (CHI+vehicle) and (CHI+500 mg/kg Pg) at the indicated time point. Mixed-effects analysis followed by Tukey-Kramer multiple comparison tests.

FIG. 2 is a graph showing motor assessment in an Open Field test in the following groups of mice: control mice which were not traumatized and not treated (Sham), control mice which were not traumatized and were treated with the vehicle (Sham+vehicle), CHI+vehicle, CHI+500 mg/kg Pg oil, and CHI+200 mg/kg Pg oil, 3 weeks post injury. Sham n=5 round-1, Sham+vehicle n=18 rounds-3, CHI+vehicle n=25 rounds-4, CHI+500 mg/kg Pg oil: n=17 rounds-2, and CHI+200 mg/kg Pg oil: n=20 rounds-3. Results are presented as mean±SD. One-Way ANOVA followed by Tukey correction. FIG. 2A shows measurements of the total path (m). FIG. 2B shows measurements of the average speed (m/sec).

FIG. 3A is a graph showing escape latency in seconds (sec) in a Barnes maze (BM) test performed two weeks after the trauma, and 3 days from the end of treatment with Pg oil. The mice were tested at 4 consecutive time points after the acquisition period: 1 day, 2 days, 3 days, and 4 days. The following groups were tested: Sham, Sham+vehicle, CHI+vehicle, CHI+500 mg/kg Pg oil, and CHI+200 mg/kg Pg oil. The results are a summary of 4 repetitions. Sham: n=21 rounds-4, Sham+vehicle: n=35 rounds-6, CHI+vehicle: n=41 rounds-6, CHI+500 mg/kg Pg oil: n=33 rounds-4, CHI+200 mg/kg Pg oil: n=20 rounds-2. Results are presented as mean±SEM. [*] indicates a P-value of <0.05, [***] indicates a P-value of <0.001 and [****] indicates a P-value of <0.0001. The P-values refer to the comparison of the groups (CHI+vehicle) and the treatment groups (CHI+200 mg/kg Pg oil or CHI+500 mg/kg Pg) at the indicated time point. Mixed-effects analysis followed by Fisher's LSD test.

FIG. 3B is a graph showing escape latency in seconds (sec) in a Barnes maze (BM) recall test (Recall 2 weeks) performed 24 hours (24 h) and 7 days (7 d) after completion of learning (7 days and 14 days from the end of treatment with Pg oil, respectively) for memory assessment. The following groups were tested: Sham n=21 rounds-4, Sham+vehicle: n=35 rounds-6, CHI+vehicle: n=41 rounds-6, CHI+500 mg/kg Pg oil: n=33 rounds-4, CHI+200 mg/kg Pg oil: n=20 rounds-2. Results are presented as mean±SEM. Significant main effects of group (p=0.01) and time of recall (p=0.0003). [*] indicates a P-value of =0.024 (comparison of CHI+200 mg/kg Pg oil 24 h vs 7 d), [**] indicates a P-value of <0.01 (comparison of CHI+vehicle 24 h vs. 7 d). For Sham groups and CHI+500 mg/kg Pg oil no significant differences between 24 h and 7 days recall. a P-value of <0.01. Mixed-effects analysis followed by Fisher's LSD test.

FIG. 4A is a graph showing escape latency in seconds (sec) in a Barnes maze (BM) test performed 2 months after the trauma. The mice were tested at 4 consecutive time points after the acquisition period: 1 day, 2 days, 3 days, and 4 days. The following groups were tested: Sham, Sham+vehicle, CHI+vehicle, CHI+500 mg/kg Pg oil. The results are a summary of 4 repetitions. Sham n=5, Sham+vehicle n=5, CHI+vehicle n=12, CHI+500 mg/kg Pg oil n=12. Results are presented as mean±SEM. [*] indicates a P-value of <0.05 (comparison of CHI+vehicle with CHI+500 mg/kg Pg oil at various time points). Mixed-effects analysis followed by Fisher's LSD test.

FIG. 4B is a graph showing escape latency in seconds (sec) in a Barnes maze (BM) recall test 2 months after the trauma, and 1.5 months after the last treatment with Pg oil. The test was performed 24 hours (24 h) and 7 days (7 d) after completion of learning, for memory assessment. The following groups were tested: Sham n=5, Sham+vehicle n=5, CHI+vehicle n=12, CHI+500 mg/kg Pg oil n=12. Results are presented as mean±SEM. [*] indicates a P-value <0.05. Mixed-effects analysis followed by Fisher's LSD test.

FIG. 5A-5E is a schematic representation showing learning strategies in a Barnes maze (BM) test. FIG. 5A shows typical Barnes maze strategies. FIG. 5B-5E show learning strategies in BM tests performed two months after the trauma (and 1.5 months after the last treatment with Pg oil) in Sham mice (5B), Sham+vehicle mice (5C), TBI+vehicle mice (5D), and TBI+Pg oil mice (FIG. 5E).

FIG. 6A is a graph showing escape latency in seconds (sec) in a Barnes maze (BM) test performed 6 months after the trauma. The mice were tested at 4 consecutive time points after the acquisition period: 1 day, 2 days, 3 days, and 4 days. The following groups were tested: Sham, Sham+vehicle, CHI+vehicle, and CHI+200 mg/Kg Pg oil treated 2 weeks post injury plus one dose once a week for 1 year post the injury as a follow up (FU) treatment. The results are a summary of 4 repetitions. Sham n=5, Sham+vehicle n=5, CHI+vehicle n=11, CHI+200 mg/kg Pg oil (FU) n=11. Results are presented as mean±SEM. [*] indicates a P-value <0.05, [**] indicates a P-value <0.01. The P-values refer to the comparison of the groups (CHI+vehicle) and Sham group at the indicated time points. CHI+200 mg/kg Pg oil FU is not significantly different versus Sham groups at all time points. Mixed-effects analysis followed by Fisher's LSD test.

FIG. 6B is a graph showing escape latency in seconds (sec) in a Barnes maze (BM) recall test (Recall 6 months from the trauma, and 5.5 months after the last treatment with Pg oil) performed 24 hours (24 h) and 7 days (7 d) after completion of learning. The following groups were tested: Sham n=5, Sham+vehicle n=5, CHI+vehicle n=11, CHI+200 mg/kg Pg oil (FU) n=11. Results are presented as mean f SEM. Mixed-effects analysis followed by Fisher's LSD test. No significant differences were found.

FIG. 6C is a graph showing escape latency in seconds (sec) in a Barnes maze (BM) test performed 12 months after the trauma. The mice were tested at 4 consecutive time points after the acquisition period: 1 day, 2 days, 3 days, and 4 days. The following groups were tested: Sham, Sham+vehicle, CHI+vehicle, and CHI+200 mg/Kg Pg oil treated 2 weeks post injury plus one dose once a week for 1 year post the injury as a follow up (FU) treatment. The results are a summary of 4 repetitions. Sham n=5, Sham+vehicle n=5, CHI+vehicle n=11, CHI+200 mg/kg Pg oil (FU) n=11. Results are presented as mean±SEM. [*] indicates a P-value <0.05. The P-values refer to the comparison of the groups (CHI+vehicle) and (CHI+200 mg/Kg Pg oil) group at the indicated time points. Mixed-effects analysis followed by Fisher's LSD test.

FIG. 6D is a graph showing escape latency in seconds (sec) in a Barnes maze (BM) recall test performed 24 h following acquisition and 12 months after the trauma. The following groups were tested: Sham, Sham+vehicle, CHI+vehicle, and CHI+200 mg/Kg Pg oil treated 2 weeks post injury plus one dose once a week for 1 year post the injury as a follow up (FU) treatment. Sham n=5, Sham+vehicle n=5, CHI+vehicle n=11, CHI+200 mg/kg Pg oil (FU) n=11. Results are presented as mean±SEM. [*] indicates a P-value <0.05. The P-values refer to the comparison of the groups (CHI+vehicle) and (CHI+200 mg/Kg Pg oil) group at the indicated time points. One-way Anova followed by Fisher's LSD test.

FIG. 7A is a graph showing escape latency in seconds (sec) in a Barnes maze (BM) test performed 9 months after the trauma. The mice were tested at 4 consecutive time points after the acquisition period: 1 day, 2 days, 3 days, and 4 days. The following groups were tested: Sham, Sham+vehicle, CHI+vehicle, and CHI+500 mg/kg Pg oil. The results are a summary of 4 repetitions. Sham n=5, Sham+vehicle n=5, CHI+vehicle n=12, CHI+500 mg/kg Pg oil n=12. Results are presented as mean±SEM. Mixed-effects analysis followed by Fisher's LSD test. No significant differences were found.

FIG. 7B is a graph showing escape latency in seconds (sec) in a Barnes maze (BM) recall test (Recall 9 months from the trauma, and 8.5 months after the last treatment with Pg oil) performed 24 hours (24 h) and 7 days (7 d) after completion of learning. The following groups were tested: Sham n=5, Sham+vehicle n=5, CHI+vehicle n=12, CHI+500 mg/kg Pg oil n=12. Results are presented as mean±SEM. Significant main effects of group (p=0.01) and time of recall (p=0.002). [**] indicates a P-value of <0.01 (comparison of CHI+vehicle 24 h Vs. 7 days). All other groups including Sham, Sham+vehicle and CHI+500 Pg mg/kg oil showed no significant differences between 24 h and 7 days recall. Mixed-effects analysis followed by Fisher's LSD test.

FIG. 8 is a graph showing results of a Novel Object Recognition (NOR) test. Results are presented as percent of the time spent by a novel object out of the total time. The following groups were tested: Sham, Sham+vehicle, CHI+vehicle, CHI+500 mg/kg Pg oil, CHI+200 mg/kg Pg oil. The animals treated with 500 mg/kg dose were tested using two different batches of Pg oil. Sham: n=14 rounds-4, Sham+vehicle: n=34 rounds=6, CHI+vehicle: n=44 rounds-6, CHI+500 mg/kg Pg oil: n=38 rounds-4, CHI+200 mg/kg Pg oil n=15 rounds-3. Results are presented as mean±SEM. [*] indicates a P-value <0.05, [***] indicates a P-value <0.001, and [****] indicates a P-value <0.0001. Two-Way ANOVA analysis followed by Fisher's LSD test.

FIG. 9 is a graph showing Mean Preference index (PI) in a Y maze test performed one month after the trauma, and 18 days after the last treatment with Pg oil. The following groups were tested: Sham: n=19 rounds-4, Sham+vehicle: n=32 rounds=6, CHI+vehicle: n=39 rounds-6, CHI+500 mg/kg Pg oil: n=32 rounds-4, CHI+200 mg/kg Pg oil: n=19 rounds-3. Results are presented as mean±SEM. [**]indicates a P-value <0.01 and [***] indicates a P-value <0.001. One-Way ANOVA followed by Fisher's LSD test.

FIG. 10A is a graph showing escape latency in seconds (sec) in a Barnes maze (BM) acquisition test performed 2 weeks after the trauma, and 3 days after the last treatment with Citronellol. The mice were tested at 4 consecutive time points after the acquisition period: 1 day, 2 days, 3 days, and 4 days. The following groups were tested: CHI+vehicle n=3, CHI+125 mg/kg Citronellol n=5, and CHI+50 mg/kg Citronellol n=5, one round for all groups. The results are a summary of 4 repetitions. Results are presented as mean±SEM. [*] indicates a P-value <0.05 (Comparison of CHI+vehicle with the treatment groups). Of the Mixed-effects analysis followed by Fisher's LSD test.

FIG. 10B is a graph showing escape latency in seconds (sec) in a Barnes maze (BM) recall test (Recall 2 weeks) performed 24 hours (24 h) and 7 days (7 d) after completion of learning (7 days and 14 days from the end of treatment with Citronellol, respectively) for memory assessment. The following groups were tested: CHI+vehicle: n=3, CHI+125 mg/kg Citronellol: n=5, and CHI+50 mg/kg Citronellol: n=5, one round for all groups. Results are presented as mean±SEM. Mixed-effects analysis followed by Fisher's LSD test. No significant difference was detected.

FIG. 11A is a graph showing the lesion volume presented as a percent of the lesion area of the total brain in the following groups of mice: CHI+vehicle: n=10 rounds-3, CHI+500 mg/kg Pg oil: n=6 rounds-2, and CHI+200 mg/kg Pg oil: n=6 rounds-2. Results are presented as mean±SEM.

FIG. 11B is a graph showing the left ventricle volume (mm²) in the following groups of mice: Sham: n=6 rounds-2, CHI+vehicle: n=10 rounds-3, CHI+500 mg/kg Pg oil: n=6 rounds-2, and CHI+200 mg/kg Pg oil: n=6 rounds-2. Results are presented as mean±SEM.

FIG. 12A-C are graphs showing the number of cells (mean no. of neurons) in various areas of the left hemisphere hippocampus: CA1 (FIG. 12A), DG (dentate gyrus) (FIG. 12B), and CA3 (FIG. 12C), in the following groups of mice: Sham: n=7 rounds-2, Sham+vehicle: n=6 rounds-3, CHI+vehicle: n=9 rounds-3, CHI+500 mg/kg Pg oil: n=7 rounds-3, and CHI+200 mg/kg Pg oil: n=6 rounds-2. Results are presented as mean±SEM using 2 Way ANOVA followed by Fisher's LSD test. [*] indicates a P-value <0.05, [**] indicates a P-value <0.01, and [***] indicates a P-value <0.001.

FIG. 13A-C are graphs showing the number of cells (mean no. of neurons) in various areas of the right hemisphere hippocampus: CA1 (FIG. 13A), DG (FIG. 13B), and CA3 (FIG. 13C), in the following groups of mice: Sham: n=7 rounds-2, Sham+vehicle: n=6 rounds-3, CHI+vehicle: n=9 rounds-3, CHI+500 mg/kg Pg oil: n=7 rounds-3, and CHI+200 mg/kg Pg oil: n=6 rounds-2. Results are presented as mean f SEM using 2 Way ANOVA followed by Fisher's LSD test. [*] indicates a P-value <0.05, [**] indicates a P-value <0.01, and [***] indicates a P-value <0.001.

FIG. 14 is a graph showing GFAP staining intensity (% mean intensity) in the ipsilateral cortex. The following groups were tested: Sham n=4, Sham+vehicle n=3, CHI+vehicle n=7, CHI+500 mg/kg Pg oil n=7, and CHI+200 mg/kg Pg oil n=4; 2 rounds for all groups. Results are presented as percentage mean±SEM. [*] indicates P<0.05, [***] indicates a P-value <0.001, and [****] indicates a P-value <0.0001. One-Way ANOVA followed by Fisher's LSD test.

FIG. 15A-C are graphs showing the number of cells as determined by measuring NeuN staining in ipsilateral cortex regions: frontal cortex (15A), forelimb cortex (15B) and parietal cortex (15C). The following groups were tested: Sham n=3, CHI+vehicle n=3, and CHI+500 mg/kg Pg oil n=5, one round for all groups. Results are presented as mean±SEM. [**] indicates a P-value <0.01.

FIG. 16A-C are graphs showing the number of cells as determined by measuring NeuN staining in contralateral cortex regions: contra frontal cortex (16A), contra forelimb cortex (16B) and contra parietal cortex (16C). The following groups were tested: Sham n=3, CHI+vehicle n=3, and CHI+500 mg/kg Pg oil n=5, 1 round for all groups. Results are presented as mean±SEM. [*] indicates a P-value <0.05.

FIG. 17A-C are graphs showing mean intensity of NF200 staining in ipsilateral cortex regions: frontal cortex (17A), forelimb cortex (17B) and parietal cortex (17C). The following groups were tested: Sham n=3, CHI+vehicle n=3, and CHI+500 mg/kg Pg oil n=5, one round for all groups. Results are presented as mean±SEM. P-values are indicated in the graph.

FIG. 18A-C are graphs showing mean intensity of NF200 in contralateral cortex regions: contra frontal cortex (18A), contra forelimb cortex (18B) and contra parietal cortex (18C). The following groups were tested: Sham n=3, CHI+vehicle n=3, and CHI+500 mg/kg Pg oil n=5, one round for all groups. Results are presented as mean±SEM. P-values are indicated in the graph.

FIG. 19A-B are graphs showing the mean number of cells which express CREB in the ipsilateral (19A) and the contralateral (19B) cortex. The following groups were tested: Sham n=3, CHI+vehicle n=3, and CHI+500 mg/kg Pg oil n=5, one round for all groups. Results are presented as mean±SEM. [*] indicates a P-value <0.05. In FIG. 19A the P-values are indicated in the graph.

FIG. 20A-D describe microglia branch length and endpoints. FIGS. 20A and 20B are illustrations of skeletonized microglia. 20A—sham operated mouse; 20B—cortex lesion of a CHI mouse. FIG. 20C and FIG. 20D are graphs showing the branch length/cell (μm) (20C) and the no. of endpoints/cell (20D) of Iba-1 positive cells, in the lesion cortex region. The following groups were tested: Sham n=5, CHI+vehicle n=4, and CHI+500 mg/kg Pg oil n=5, one round for all groups. Results are presented as mean±SEM. [*] indicates a P-value <0.05, [**] indicates a P-value <0.01, [***] indicates a P-value <0.001, and [****] indicates a P-value <0.0001.

FIG. 21A-B are graphs showing a cell count (no. of cells) of microglia positively stained with Iba-1 (21A), and the percent of microglia that are double positive (CD206+ and Iba-1+) of the total no. of Iba-1+ cells (21B), in the lesioned region of the left cortex. Sham n=5, CHI+vehicle n=4, and CHI+500 mg/kg Pg oil n=5, one round for all groups. Results are presented as mean±SEM. [*] indicates a P-value <0.05, [**] indicates a P-value <0.01, and [****] indicates a P-value <0.0001.

FIG. 22. is a graph showing time-course (hours) of phagocytosis of pHrodo red E. coli bioparticles by BV2 mouse microglial cells in the presence of 20 μg/ml Pg oil or in the presence of vehicle. Phagocytosis has been quantified as the total integrated fluorescence for each time-point, mean±SEM, n=3 wells.

FIG. 23 is a graph showing relative (%) TNFα levels in cortical protein taken from the left cortex, 24 hours after TBI. Mice were treated for 2 days before TBI and at the day of TBI (total of 3 treatments) with 500 mg/kg Pg oil in the following groups of mice: Sham+vehicle, TBI+vehicle, and TBI+Pg. Results are presented as mean±SEM of two independent experiments, a total of 10 animals were included in each treatment group (5 mice in each experiment). TNFα levels were determined by ELISA. Each animal was tested in duplicate using 180 μg of cortical protein. TNFα levels in the sham+vehicle group were considered as 100%, 100%=42.72 μg TNFα/μg protein. ANOVA followed by Tukey-Kramer multiple comparison tests. *p<0.05.

FIGS. 24A and 24B are graphs showing TNFα pg/μg cortical protein taken from the left cortex (FIG. 24A) or hippocampal protein taken from the left hippocampus (FIG. 24B), 11 days after TBI. Mice were treated 9 times, starting one day after TBI, with 500 mg/kg Pg oil in the following groups of mice: Sham+vehicle, Sham without vehicle, TBI+vehicle, and TBI+Pg oil. Results are presented as mean±SEM of all the animals in each treatment group (3-4 animals in each treatment group). The results represent a single in vivo experiment that was tested in duplicates. 180 μg of cortical protein and 159 μg of hippocampal protein were used to determine TNFα levels by ELISA. ANOVA followed by Tukey-Kramer multiple comparison tests. ***p<0.001.

FIG. 25 is a graph showing TNFα pg/μg cortical protein, 30 days after TBI. Mice received 9 treatments with 500 mg/kg Pg oil starting one day after TBI. The mice received treatment every day or every three days for a total amount of 9 treatment spread over 12 days in the following groups of mice: Sham+vehicle, TBI+vehicle, and TBI+Pg. Results are presented as mean±SEM of all the animals in each treatment group (4 animals in each treatment group). A single in vivo experiment that was tested in duplicates. 180 μg of cortical protein was used to determine TNFα levels by ELISA. ANOVA followed by Tukey-Kramer multiple comparison tests. ***p<0.001.

FIGS. 26A and 26B are graphs showing IL-1β levels in the left cortex (FIG. 26A: pg/μg cortical protein, 2 in vivo experiments), and in the left hippocampus (FIG. 26B: pg/μg hippocampal protein, 1 in vivo experiment) 11 days after TBI in the following experimental groups of mice: Sham (with and without vehicle), TBI+vehicle, and TBI+Pg oil. Mice were treated 24 hours after TBI, total of 9 treatments during 12 days after TBI. Results are presented as mean±SEM of all the animals in each treatment group (n=3 in each group). Each animal was tested in duplicates. 180 μg of cortical homogenate and 30 μg of hippocampal homogenate were used to determine IL-1p levels by ELISA. ANOVA followed by Tukey-Kramer multiple comparison tests. *p<0.05, **p<0.01, ***p<0.001.

FIG. 26C is a graph showing IL-1β levels in the left cortex (pg/μg cortical protein), one month after TBI in the following experimental groups of mice: Sham (with and without vehicle), TBI+vehicle, and TBI+Pg oil (500 mg/kg). Mice were treated 24 hours after TBI, total of 9 treatments during 12 days after TBI. Results are presented as mean±SEM of all the animals in each treatment group (n=4 in each group). Each animal was tested in duplicates. 45 μg of cortical homogenate was used to determine IL-10 levels by ELISA. ANOVA followed by Tukey-Kramer multiple comparison tests.

FIG. 27 is a graph showing the relative levels of IL-1β in hippocampal protein taken from the left hippocampus, 48 hours after TBI. Mice were pretreated with 500 mg/kg Pg oil for 2 days before TBI, and at the day of TBI (total of 3 treatments), in the following groups of mice: Sham+vehicle, TBI+vehicle, and TBI+Pg oil. Results are presented as mean±SEM of all the animals in each experimental group (n=5 in each group). Each animal was tested in duplicate using 30 μg of hippocampus homogenate. IL-1β levels in the sham+vehicle group were considered as 100%, 100% is 121.33 μg IL-1β/μg protein. ANOVA followed by Tukey-Kramer multiple comparison tests. *P<0.05.

FIG. 28 is a graph showing IL-1β pg/μg cortical protein, 30 days after TBI. Mice were treated one day after TBI (9 treatments in 12 days) in the following groups of mice: Sham, TBI+vehicle, and TBI+200 mg/kg Pg oil. Results are presented as mean±SEM of all the animals in each treatment group (3-5 animals in each treatment group). A single in vivo experiment that was tested in duplicates. 45 μg of cortical homogenate was used to determine IL-1p levels by ELISA. ANOVA followed by Tukey-Kramer multiple comparison tests. **P<0.01; ***P<0.001.

FIG. 29 is a graph showing IL-6 levels (pg/μg cortical protein), 24 hours after TBI. Mice were pretreated for 2 days before TBI, and at the day of TBI (total of 3 treatments) in the following groups of mice: Sham+vehicle, TBI+vehicle, and TBI+500 mg/kg Pg oil. Results are presented as mean±SEM of all the 10-12 animals in each treatment group (n=10, in two in vivo experiments, 5 animals in each treatment group). Each animal was tested in duplicates. 90 μg of cortical homogenate was used to determine IL-6 levels by ELISA. ANOVA followed by Tukey-Kramer multiple comparison tests. ***P<0.001.

FIG. 30 is a graph showing relative (%) IL-6 levels in cortical protein taken from the left cortex, 48 hours after TBI. Pg oil was administered 2 days before TBI and at the day of TBI (total of 3 treatments), in the following groups of mice: Sham+vehicle, TBI+vehicle, and TBI+500 mg/kg Pg oil. Results are presented as mean±SEM of all the 10-12 animals in each treatment group (n=10, in two in vivo experiments, 5 animals in each treatment group). Each animal was tested in duplicate. 90 μg of cortical homogenate was used to determine IL-6 levels by ELISA. IL-6 levels in “sham+vehicle” group were considered as 100%, 100% is 43.94 μg IL-6/pg protein. ANOVA followed by Tukey-Kramer multiple comparison tests. *p<0.05, **p<0.01.

FIG. 31 is a graph showing acetyl choline esterase (AchE) activity (mU/mL) in the cortex, 11 days after TBI in the following groups of mice: Sham, TBI+vehicle, and TBI+500 mg/kg Pg oil. Results are presented as mean±S.D. for one animal in each treatment group tested in duplicate. 100 μg of cortical homogenate was used.

FIG. 32 is a graph showing lipid peroxidation (μM MDA) in the cortex, 30 days after TBI in the following groups of mice: Sham, and TBI+0, 200, or 500 mg/kg Pg oil. Results are presented as mean±SEM. Four mice were tested from each experimental group. Each mouse was tested in 4 replicates. 190 μg of cortical protein was used. ANOVA followed by Tukey-Kramer multiple comparison tests. ***p<0.001.

FIG. 33 is a graph showing the ratio of phosphorylated CREB/Total CREB in the cortex and hippocampus 30 days after TBI in the following groups of mice: TBI+vehicle and TBI+Pg oil. Results are presented as mean±S.D. One in vivo experiment, 3-4 mice/group. Students t-test *p<0.05.

### DETAILED DESCRIPTION OF EMBODIMENTS

The present invention is based on the surprising finding that feeding mice before or after a closed head injury (an animal model of TBI) with essential oil extracted from the plant Pelargonium graveolens (Pg) significantly improved their learning and memory performance and reduced their anxiety in immediate (short-term) and long-term behavioral tests, as well as various biochemical parameters in the cortex and hippocampus.

Therefore, in a first of its aspects, the present invention provides a pharmaceutical or nutritional composition for treating, preventing, or ameliorating the symptoms of traumatic brain injury in a subject, the composition comprising essential oil extracted from Pelargonium graveolens and one or more physiologically acceptable carriers.

As used herein the term “pharmaceutical composition” refers to a composition comprising Pelargonium graveolens (Pg) essential oil, wherein said composition is provided as a medicament. As used herein the term “nutritional composition” refers to a composition comprising Pelargonium graveolens (Pg) essential oil, wherein said composition is provided as a food supplement, nutrition additive, food, or beverage. In some embodiments, the compositions of the invention are enriched by the addition of an active compound, e.g., citronellol, thereby increasing the concentration of the active compound, e.g., citronellol in the composition.

As used herein the term “Pelargonium graveolens (Pg)” is a pelargonium species which was originally grown in South Africa but can be found now all around the world.

The terms “Pelargonium graveolens (Pg) essential oil” or “Pelargonium graveolens (f) oil” are used interchangeably herein and refer to an extract of the Pg plant foliage. The essential oil can be extracted from the plant foliage using any method known in the art, for example using a steam or hydro-distillation apparatus (see e.g., Elmann et al [9] or [10] which is incorporated herein by reference). A non-limiting example of a distillation apparatus is a hydro-distillation Clevenger apparatus system.

The essential oil extracted from the plant Pelargonium graveolens is a defined mixture of phytochemicals [see for example 11, 12]. In one embodiment, the content of the essential oil can be identified by Gas Chromatography-Mass Spectrometry (GC-MS). Non-limiting examples of the essential oil content are provided in Example 1, Table 1. It is characterized by low molecular weight molecules (˜150). Due to this low molecular weight and lipophilicity of the essential oil components, they can cross the blood-brain barrier and affect processes and cells in the brain.

The essential oil of Pg is defined by the FDA as GRAS (generally recognized as safe) and therefore it is safe for consumption and use in the food industry.

The terms “treating”, “preventing”, or “ameliorating” is used conventionally and refers to the management or care of a subject for the purpose of combating, alleviating, reducing, relieving, or improving a subject's traumatic brain injury, or any symptom thereof. The term also encompasses prophylactic treatment of subjects at risk of TBI. The terms encompass any reduction in the subject's traumatic brain injury, or any symptom thereof as evidenced, for example, by measurement of cognitive, physiological, radiological (e.g., by Magnetic Resonance Imaging (MRI)), and/or clinical indicators of brain injury.

As used herein the term “Traumatic Brain Injury (TBI)” refers to a disruption in the normal function of the brain that is caused by an external force such as a direct impact on the head (blow, bump, or jolt), rapid acceleration, blast waves, or penetration of the skull that reaches brain tissue. TBI may be associated with characteristic cognitive, emotional, physical, social, and behavioral symptoms indicating alteration in the normal brain function: loss of or decreased consciousness, loss of memory (amnesia), focal neurological deficits such as muscle weakness, loss of vision, change in speech, and/or alteration in mental state such as disorientation, slow thinking, difficulty in concentrating, sleep disorders, anxiety, mood disorders and/or social interaction.

The insult severity of TBI can be mild (e.g., a concussion), moderate, or severe, depending on the extent of damage to the brain. Mild cases may result in a brief change in mental state or consciousness followed by complete recovery. Severe cases may result in permanent disability, extended periods of unconsciousness, coma, or even death.

As shown in the Examples below treatment of mice suffering from closed head injury (as a model for traumatic brain injury) with the Pg oil of the invention, resulted in remarkable improvement of cognitive functions short term and long term after the trauma.

Accordingly, the present invention also provides a pharmaceutical or nutritional composition for improving cognitive function of a subject suffering from traumatic brain injury, the composition comprising essential oil extracted from Pelargonium graveolens and one or more physiologically acceptable carriers.

As used herein the term “improving” refers to any amelioration, elimination, remedy or slowing down of the cognitive decline which occurs in a subject suffering from TBI. Improvement can be manifested for example in an increased ability to learn, remember, or concentrate. Multiple tests are known in the literature for assessing the cognitive functions of subjects suffering from TBI. Any of these tests may be employed to assess the improvement in the subject's cognitive state or functions, and it is in the physician's discretion to select the most appropriate tool. Non-limiting examples include the MoCA (Montreal Cognitive Assessment) test, which is mostly used for detecting mild cognitive impairment, the MMSE (mini-mental state examination) test which is mostly used for finding more serious cognitive impairment, Mini-Cog evaluation, and others.

In one embodiment, the present invention provides a pharmaceutical or nutritional composition for improving long-term cognitive function of a subject suffering from traumatic brain injury, the composition comprising essential oil extracted from Pelargonium graveolens and one or more physiologically acceptable carriers.

As used herein the term “long-term” refers to the ability of the treatments to affect the cognitive functions not only immediately after the trauma but for longer periods of time. The term may refer to several months after the trauma, for example, but not limited to two months, six months, nine months, 12 months or more.

The compositions of the invention may be administered as such, or may be incorporated into food products, beverages (e.g., juices) or combined with commonly used food additives such as corn syrup.

In one embodiment, the composition of the invention is a nutritional composition administered by feeding.

In other embodiments, the composition of the invention is a pharmaceutical composition that can be administered and dosed in accordance with good medical practice. For example, the pharmaceutical composition can be introduced to the body by any suitable route including oral, intraperitoneal, subcutaneous, transcutaneous, topical, intramuscular, intraarticular, subconjunctival, or mucosal, e.g., intranasal, or intraocular administration. The compositions may be administered systemically or may be locally administered. Local administration may be facilitated by using an implant that acts to retain the active dose at the site of implantation. The active agent may be formulated for immediate activity, or it may be formulated for sustained release.

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, formulations are prepared by uniformly and intimately bringing into association the active ingredients of the invention with liquid carriers or finely divided solid carriers or both, and then, if necessary, shaping the product.

The compositions may be formulated into any of many possible dosage forms such as, but not limited to, tablets, capsules, liquid syrups, nasal spray, nasal drops, soft gels, suppositories, patches, and enemas.

The Pg oil extract may be diluted to a desired final concentration by mixing with an appropriate vehicle solution, e.g., an oily solution, including but not limited to any plant-derived essential oil or medium chain triglycerides (MCT). In one embodiment the Pg oil is diluted in olive oil. As used in the Examples section below, the term “vehicle” refers to olive oil given to the animals as a control without the active agent.

In addition to the ingredients particularly mentioned above, the formulations may also include other agents conventional in the art having regard to the type of formulation in question.

Still further, the compositions of the invention and any components thereof may be applied as a single daily dose or multiple daily doses.

The compositions may be administered for a treatment session of between about 5 days to about four weeks or more immediately after the trauma. In one embodiment, the compositions are administered daily or once every two or three days for a period of 10 to 14 days immediately after trauma. In one embodiment, a total number of between 5 treatments and 15 treatments is provided following the trauma starting immediately after trauma (namely within minutes or hours), or one day after the trauma. A person skilled in the art would realize that the sooner the first treatment is given, the better. Therefore, in specific embodiments a total number of 5, or 6, or 7, or 8, or 9, or 10, or 11, or 12, or 13, or 14, or 15, or more daily treatments are provided to the subject. In a specific embodiment the treatment session comprises between 8 and 12 daily treatments. In another specific embodiment the treatment session comprises 9 daily treatments. In some embodiments, one or more additional treatment sessions may be given to the subjects between about one month and about one year after the trauma. In one embodiment, the additional treatment session comprises between 8 and 12 daily treatments given over a period of about two weeks. In one embodiment, the additional treatment session is given 9 months after the injury and comprises about 9 daily treatment doses given over a period of about two weeks. In one embodiment, the “daily treatment” refers to one administration of Pg oil per day. In other embodiments, the “daily treatment” refers to two or three administrations of Pg oil per day.

In another embodiment, the compositions are administered as a prophylactic treatment for subjects at risk of head trauma or TBI, e.g., soldiers, policemen, fire fighters, or athletes. In a specific embodiment, the subject receives between 1 and 5 daily treatments commencing several days prior to the intended risky event. In one embodiment, the subject receives a total amount of three daily treatments commencing two days prior to the risky event.

In another embodiment, the compositions are administered for an unlimited time-period (also referred to as chronic administration), for example for one year, two years or more. In one embodiment, after the initial intensive treatment session, the compositions may be given once a week for as long as desired (e.g., for one year, two years, or more), also referred to herein as “chronic treatment”. For example, the compositions may be given as a food supplement for regular consumption.

The composition of the invention can be administered alone, or in combination with other active agent(s). In certain embodiments the additional active agents include, but are not limited to, citronellol, 3,5,4′-trihydroxy-6,7,3′-trimethoxyflavone (TTF), or steroids.

The compositions of the present invention may be combined with other types of treatments including behavioral therapy, rehabilitation therapy, diet restrictions and pharmacological intervention.

In another aspect, the present invention provides a method of treating, preventing, or ameliorating the symptoms of TBI, the method comprising administering to a subject in need thereof a therapeutically effective amount of Pelargonium graveolens essential oil.

By the term “administering”, it is meant that the composition is delivered to a subject by any means or route which is effective to achieve the desired result, including e.g., oral, parenteral, enteral, intraperitoneal, topical, transdermal (e.g., using any standard patch), subcutaneous, intravenous, intra-arterial, intramuscular, buccal, sublingual, ophthalmic, nasal, by aerosol, by inhalation, rectal, vaginal, and intrathecal.

In some embodiments the composition of the invention is administered to human subjects in an amount of between about 1 mg/kg to 50 mg/kg per day or once every two or three days, depending upon the subject's physical condition, the severity of disease, mode of administration, etc. In specific embodiments the composition of the invention is orally administered to human subjects in an amount of about 16 mg/kg (which approximately corresponds to an amount of 200 mg/kg in mice) or about 40 mg/kg (which approximately corresponds to an amount of 500 mg/kg in mice) per day, or once every two or three days. In one embodiment the composition of the invention is administered to human subjects in an amount of about 41 mg/kg daily or once every two or three days. In one embodiment the composition of the invention is administered to human subjects intranasally in an amount of between about 1 mg/kg and 5 mg/kg (e.g., 1.6 or 4.1 mg/kg) daily or once every two or three days. In one embodiment the composition of the invention is administered to human subjects by inhalation in an amount of between about 0.1% and 10% (% of essential oil per inhalation volume) daily or once every two or three days. The compositions can be administered at any suitable time, e.g., prior to or after a meal, prior to activity, prior to sleeping and at different times of the day, e.g., in the morning, in the evening etc.

In another aspect, the present invention provides a pharmaceutical or nutritional composition for treating, preventing, or ameliorating the symptoms of traumatic brain injury in a subject, the composition comprising isolated citronellol and one or more physiologically acceptable carriers.

In another aspect, the present invention provides a method of treating, preventing, or ameliorating the symptoms of TBI, the method comprising administering to a subject in need thereof a therapeutically effective amount of isolated citronellol.

In some embodiments the composition comprising isolated citronellol is administered to human subjects in an amount of between about 1 mg/kg to 15 mg/kg per day or once every two or three days. In specific embodiments the composition of the invention is orally administered to human subjects in an amount of about 4 mg/kg (which approximately corresponds to an amount of 50 mg/kg in mice) or about 10 mg/kg (which approximately corresponds to an amount of 125 mg/kg in mice) per day, or once every two or three days. In one embodiment the treatment session comprises between 5 and 15 treatments over a period of between about 10 days and 16 days, e.g., 9 treatments over a period of 14 days.

In another embodiment, the composition is administered for an unlimited time-period (also referred to as chronic administration), for example for one year, two years or more. In one embodiment, after the initial intensive treatment session, the composition may be given once a week for as long as desired (e.g., for one year, two years, or more), also referred to herein as “chronic treatment”.

In one embodiment the composition of the invention is administered to human subjects intranasally in an amount of between about 0.1 mg/kg and 1.5 mg/kg (e.g., 0.4 or 1.0 mg/kg) daily or once every two or three days.

### EXAMPLES

### Example 1

### Preparation of the Pg Essential Oil

Plant material production: Pg varieties from the Newe Ya'ar living collection were vegetatively propagated (by rooting cut stems) and grown in experimental plots at the Newe Ya'ar Research Center. Fertigation was done by a dripping system.

Essential oil production: Steam-distillation was carried out in an essential oil pilot plant using a Clevenger apparatus system. 30 kg of fresh leaves and stems were distilled for 2.0 h in a 130 L container. After extraction, the oil was cooled and separated from the aqueous phase. The essential oil collected and stored in darkness at 4-6° C., to avoid rapid oxidation.

Essential oil analysis: Qualitative and quantitative analysis of the volatile compounds of essential oil was carried out using a gas chromatograph-mass spectrometer (GC/MS) (Agilent Technologies, Palo Alto, CA, USA) equipped with an auto-sampler Combi PAL (CTC Analytic, CH-zwingen) and a Rtx-5SIL MS cap. column (95% dimethyl/5% diphenyl polysiloxane, 30 m×0.25 mm i.d.×0.25 Pm film thickness). The identification of volatile compounds was based on the comparison of the mass spectra, performed with MSDChem software (Agilent), with NIST/WILEY libraries, and relative retention indexes.

The composition of two separate preparations of the Pg oil (designated ND-4100 and ND-4482) is shown in Table 1.

TABLE 1

| | ND-4100 | ND-4482 |
|---|---|---|
| | Pg oil | Pg oil |
| NAME | % | % |
| β-Citronellol | 29.0 | 22.5 |
| Linalool | 7.4 | 9.7 |
| Geraniol | 8.7 | 7.9 |
| Citronellyl formate | 9.2 | 7.1 |
| Iso-Menthone | 6.6 | 7.0 |
| 10-epi-γ-Eudesmol | 7.5 | 4.1 |
| Geranyl formate | 2.5 | 2.4 |
| α-Terpineol | 0.9 | 1.8 |
| Germacrene D | 2.0 | 1.7 |
| β-Bourbonene | 1.4 | 1.6 |
| Geranyl tiglate | 1.5 | 1.4 |
| E-Citronellyl tiglate | 2.2 | 1.3 |
| Geranyl propanoate | 1.1 | 1.3 |
| Caryophyllene E | 0.9 | 1.3 |
| δ-Cadinene | | 1.2 |
| Geranyl butanoate | 1.2 | 1.2 |
| Viridiflorene | 1.2 | 1.2 |
| α-Pinene | 0.6 | 1.2 |
| Phenyl ethyl tiglate | 1.3 | 1.1 |
| E-β-Ocimene | 0.3 | 1.0 |
| trans-Calamenene | | 0.9 |
| cis-Rose oxide | 1.5 | 0.9 |
| Menthone | 0.4 | 0.9 |
| α-Copaene | 0.8 | 0.7 |
| Limonene | 0.3 | 0.7 |
| trans-Muurolα-3,5-diene | | 0.7 |
| Z-β-Ocimene | 0.2 | 0.6 |
| Terpinolene | 0.2 | 0.6 |
| Myrcene | 0.2 | 0.6 |
| Geranyl acetate | | 0.6 |
| Geranial | 0.5 | 0.5 |
| Citronellyl propanoate | 0.5 | 0.5 |
| 6,9-Guaiadiene | 0.4 | 0.5 |
| allo-Aromadendrene | 0.4 | 0.5 |
| Citronellyl acetate | 0.5 | 0.4 |
| trans-Rose oxide | 0.4 | 0.4 |
| trans-Cadina-1(6),4- | 0.4 | 0.4 |
| diene | | |
| α-Humulene | 0.2 | 0.4 |
| trans-Cadina-1,4-diene | | 0.4 |
| Neral | tr | 0.4 |
| α-Phellandrene | 0.1 | 0.3 |
| Hinesol | | 0.3 |
| δ-Amorphene | | 0.2 |
| t-Cadinol | | 0.2 |
| α-Agarofuran | | 0.2 |
| γ-Eudesmol | 0.2 | 0.2 |
| α-Muurolene | 0.1 | 0.2 |
| p-Cymene | 0.1 | 0.2 |
| β-Copaene | | 0.2 |
| α-Cubebene | | 0.2 |
| β-Phellandrene | | 0.2 |
| β-Elemene | | 0.2 |
| δ-Selinene | | 0.2 |
| γ-Muurolene | | 0.2 |
| β-Selinene | | 0.1 |
| α-Gurjunene | | 0.1 |
| γ-Terpinene | | 0.1 |
| Myrcenol | | 0.1 |
| neo-Menthol | 0.1 | 0.1 |
| α-Calacorene | 0.1 | 0.1 |
| α-Terpinene | | 0.1 |
| 1-epi-Cubenol | | 0.1 |
| α-Ylangene | | 0.1 |
| p-Cymenene | | 0.1 |
| Citronellal | | 0.1 |
| 1,8-Cineole | | 0.1 |
| Citronellyl butanoate | 1.3 | |
| δ-Cadinene | 1.1 | |
| Agarospiral | 0.3 | |
| γ-Cadinene | 0.2 | |
| Camphene | | tr |
| cis-Dihydroagarofuran | | tr |
| Total | 95.9 | 93.6 |

### Example 2

Testing the Efficacy of Treatment with the Pg Essential Oil on Short-Term and Long-Term Cognitive Function in an Animal Model of Closed Head Injury (CHI)

### Animals and Ethical Statement

This study was approved by the Institutional Animal Ethics Committee of the Sheba Medical Center and complied with the guidelines of the National Research Council Guide for the Care and Use of Laboratory Animals (NIH approval no. OPRR-A5011-01,). Male C57BL/6JOlaHsd mice (8-9 weeks old) weighing 20-25 grams were purchased from Envigo, Israel, and used in all experiments. The mice were maintained under a controlled 12 hours (h) light/12 h dark cycle, with food and water provided ad libitum.

### Closed Head Injury (CHI) Model

Experimental CHI was induced by using a modified weight drop device (Chen et al [13]; Flierl et al [14]). Briefly, under 3% isoflurane anesthesia and supplementary oxygen (confirmed by the loss of response to pinch of paw), a midline longitudinal incision is performed, and the skull is exposed. A Teflon tipped cone (2 mm diameter) is placed upside down 2 mm lateral to the midline and 2 mm posterior to the bregma in the mid-coronal plane. The head is held in place and a 95 g weight is allowed to free-fall on the cone from a pre-established height, resulting in focal injury to the left hemisphere. The height of the free-fall weight is determined by the weight of the mouse and the desired CHI severity. In the present experiments, a free-fall of 6.5-7 cm was selected to induce a moderate neurological severity injury (NSS of 6-7 at 1 hr post injury). Sham mice were anesthetized and subjected to the same skin incision as described above, with no further trauma. After CHI and recovery from anesthesia (within −2 mins) the mouse is oxygenated for 30 sec (via a mask) with 95:5 oxygen: C02 and returned to its home-cage. At 1 h after CHI, the NSS is evaluated. Mice with a severity score of 9 or 10 were excluded from the study. Analgesia was achieved with dipyrone—500 mg in 250 ml drinking water provided after injury for id. Animals displaying apnea were monitored carefully and returned to their cage when adequate oxygenation was restored.

### Pg Oil Treatment:

Mice were divided into 7 different groups:
TBI mice (mice that were traumatized, i.e., subjected to traumatic brain injury (TBI) in the closed head injury (CHI) model treated with 500 mg/Kg Pg oil for 2 weeks post injury
TBI mice treated with 200 mg/Kg Pg oil for 2 weeks post injury
TBI mice treated with 200 mg/Kg Pg oil for 3 days pre-injury plus 2 weeks post injury
TBI mice treated with 200 mg/Kg Pg oil for 2 weeks plus one dose once a week for 1 year post the injury
TBI mice treated only with the vehicle solution (TBI+vehicle)
Sham: mice with no head injury and no treatment with vehicle
Mice with no head injury, treated with vehicle.

Pg oil extract was diluted with olive oil to a final concentration of 26 mg/ml and 52 mg/ml for the 200 mg/Kg and 500 mg/Kg treated groups, respectively. Pg oil was freshly diluted each day during the treatment period in glass vials and was administered by oral gavage in an equivalent volume of 200 μl as the vehicle solution.

All experimental groups were followed up and subjected to the following tests:

### (i) Neurological Severity Score:

The functional status of the mice was evaluated according to the neurobehavioral score, known as the Neurological Severity Score (NSS), which consists of several tests that assess reflexes, alertness, coordination, and motor abilities. The score is a 10-point scale that is based on the presence of some reflexes and the ability to perform motor and behavioral tasks, such as beam walking, beam balance, and spontaneous locomotion (Beni-Adani et al., 2001 [15]; Flierl et al., 2009 [14]). Animals are awarded one point for failure to perform a task, the NSS thus increasing with the severity of dysfunction. The NSS obtained 1 hr after CHI reflects the initial severity of injury (Tsenter et al., 2008 [16]).

### (ii) Cognitive Evaluation, Including the Following Behavioral Tests:

### Barnes Maze (BM) and Exploration Strategies:

The BM test is based on the instinct of the mouse to hide when exposed to environmental disturbances and is used to test spatial learning and memory. The maze consists of an elevated circular platform with holes in the perimeter and a small dark hidden goal box. Bright light and aversive noise (85 dB) force the animals to escape from the open platform surface to find the hole under which the dark chamber (21×22×21 cm), “target goal box,” is located. Visual cues of different colors and shapes are placed around the room. The mouse was placed under a cylindrical black start chamber at the center of the maze. After 10 secs, the chamber was lifted, the buzzer was switched on and the mouse was allowed to explore the maze for 3 min. The trial ended when the mouse had reached the target box or after 3 min had elapsed. Immediately after entering the target box, the buzzer was turned off and the mouse was allowed to remain there for 1 min. Animals underwent 4 trials/day for 4 days, with an inter-trial interval of 15 min. The recall test (probe) is performed as follows: 24 h and seven days after the final session of acquisition training, mice undergo a 3 min probe trial in which the escape tunnel is not removed from the apparatus. The probe trial is performed in a similar manner to the acquisition trials. For the probe trial, the latency to reaching the escape box first are recorded.
The Barnes maze test was performed:
1) Between days 14-17 (2 weeks) plus a recall test on days 18 and 25 after injury, for a total round of 6.
2) Between days 54-57 (2 months) plus a recall test on days 58 and 65 after injury for a total round of 2.
3) Between days 183-186 (6 months) plus a recall test on days 187 and 194 after injury for a total round of 2.
4) Between days 277-280 (9 months) plus a recall test on days 281 and 288 after injury for a total round of 1.
The captured videos were analyzed with Ethovision XP10 software (Noldus, The Netherlands) for later evaluation of the strategies used to locate the target box. Briefly, mice utilize a sequence of different search strategies ranging from a random search (st.1), a random and serial search (st.2), a serial search (st.3) and a spatial search (st.4) to learn the location of the target box. Spatial search (st.4) was determined as such when the mouse turned to the “correct” quadrant (which included the goal box and two adjacent holes on both sides) immediately when the trial has begun. Serial search (st.3) was determined as such when the mouse looked for the goal box by exploring the holes one by one in a serial manner while unaware of its exact location. Random search (st.1) was determined as such when the mouse was neither aware of the location of the goal box nor used an efficient search strategy, such as a serial search, to locate it. Such mice usually wandered randomly in the arena. Finally, random, and serial search (st.2) was determined as such when the mouse used both a serial search and moved randomly across the arena. The distribution into strategies was performed by a blind tester. These 4 strategies are based on the method described by Fox et al. (1998) [17] and Pitts (2018) [18] with some modifications. The strategy and the time required to locate the target box represent the efficiency of the mouse's learning ability.

### Novel Object Recognition Test:

The novel object recognition (NOR test) was performed to assess short term memory 23 d after trauma. On the first day of the test, mice were placed in the testing cage (a container measuring 60×25×40 cm) for 1 h habituation. On the following day they were returned to the same testing cage with two identical objects of similar size, surface complexity and material. The cumulative time spent by each mouse in exploring the objects was recorded manually during a total of 5 min. After 4 hours each mouse was reintroduced into the cage, where one of the familiar objects was replaced with a new one. A normal mouse spends relatively more time exploring a novel object than a familiar one, attesting to its ability to remember and distinguish novel from familiar. Exploration of the object was determined as such when the mouse's nose was pointed in the direction of the object and the mouse was actively investigating it, directly touching it or being close to it. Toys were used as NOR objects (a toy car and a doll). The “old” and “new” objects were significantly different in shape, colors, texture, and material (plastic and ceramic) but not in size (about 5×5×5 cm per object) or surface complexity. The time spent exploring each of the objects (novel or familiar) was recorded and calculated as the percentage of time from a total of 5 min.
The NOR test was performed on 7 groups: TBI+200 mg/Kg Pg oil n=20, TBI+200 mg/Kg Pg oil Pre and Post treatment n=7, TBI+500 mg/Kg Pg oil n=38, TBI+vehicle n=45, Sham+vehicle n=35 and Sham n=21.

### Open Field Test:

The open field Test is used to assess anxiety-like behavior, velocity, and locomotion. This test is based on the tendency of mice in a state of anxiety to avoid open and exposed areas and to remain close to the periphery, whereas animals in a lower state of anxiety show interest and explore the area more freely. For this purpose, mice were placed in a square white Perspex box arena (size, 50×50×30 cm). A smaller center zone was defined as 50% of the arena. The mice were allowed to explore the enclosure for 10 min. Behavioral performance, including area preference (center vs periphery), velocity and locomotion, was tracked and analyzed with EthoVision XP10 software (Noldus, Wageningen, The Netherlands).

### Y Maze Test:

At 29 days post CHI, the Y maze spatial memory test was preformed to evaluate short term spatial memory. The maze is designed as three black Perspex arms at a 120° angle from one another (“start,” “other,” and “new” arms). A mouse is placed at the “start” arm and allowed to explore freely this arm and the “other” arm for 5 min, while the “new” arm remains closed. After 2 min the mouse is returned to the maze and allowed to explore all 3 arms for 2 min. The amount of time spent in each arm is documented. Short-term memory is reflected by the novelty ratio (NR), calculated as the amount of time spent in the new arm relative to the total amount of time spent in the “other” plus “new” arms. EthoVision XP10 software (Noldus) was used to evaluate the time spent in each arm.

Finally, an experiment was performed to evaluate the effect of citronellol alone. To this purpose mice (n=5/group) were treated with two different doses of citronellol [Sigma-Aldrich](125 and 50 mg/kg). The mice received 9 daily treatments after the trauma (once every day or every three days) during a 14-days treatment session, by gavage feeding and were evaluated two weeks following the trauma in the BM test. The doses of citronellol were determined according to their relative percentage in the Pg essential oil based on the chemical analysis.

### Results

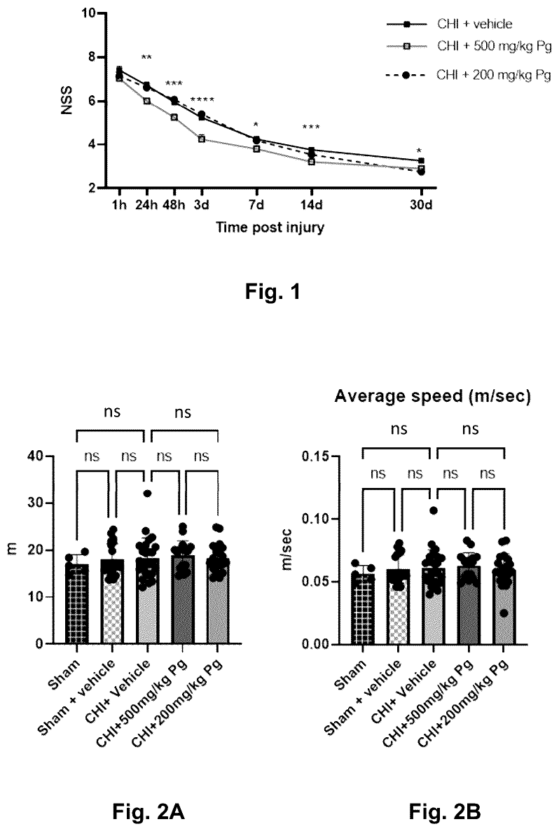

Mice were fed with Pg essential oil starting 24 hours after the trauma (closed head injury, as described in Materials and methods above) for 2 weeks, with a total of 9 daily doses. NSS evaluation to determine clinical, especially motor, function was performed 1 hour, 24 hours, 48 hours, 72 hours, 1 week, two weeks and one month (30 days) after injury. As can be seen in FIG. 1 treatment of TBI-mice (mice subjected to closed head injury, CHI) with Pg essential oil (especially with the 500 mg/kg dose) significantly reduces the neurological severity score (NSS).

In each of the behavioral tests that were performed immediately after treatment with the essential oil and up to two months afterwards, a significant improvement was found in learning and memory functions of the traumatized animals.

In the open field Test that was performed 21 days after the trauma (CHI), there were no significant differences in total path (FIG. 2A) and speed (FIG. 2B) between the tested groups, indicating no motor deficits during the behavioral study.

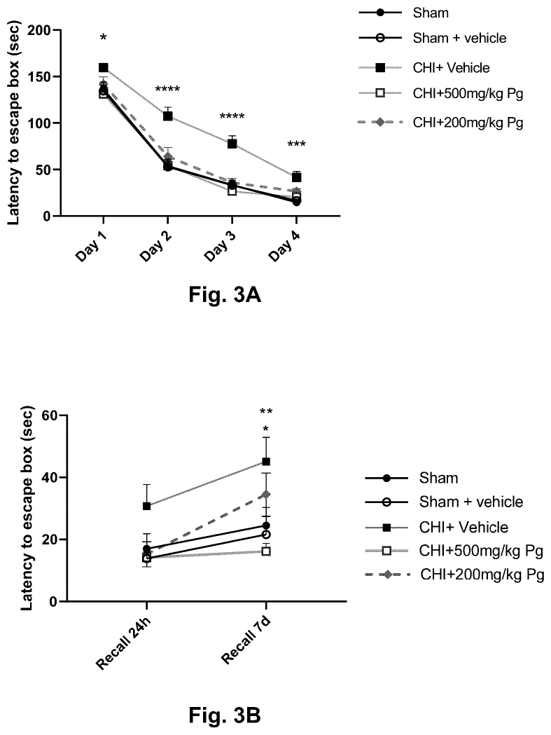

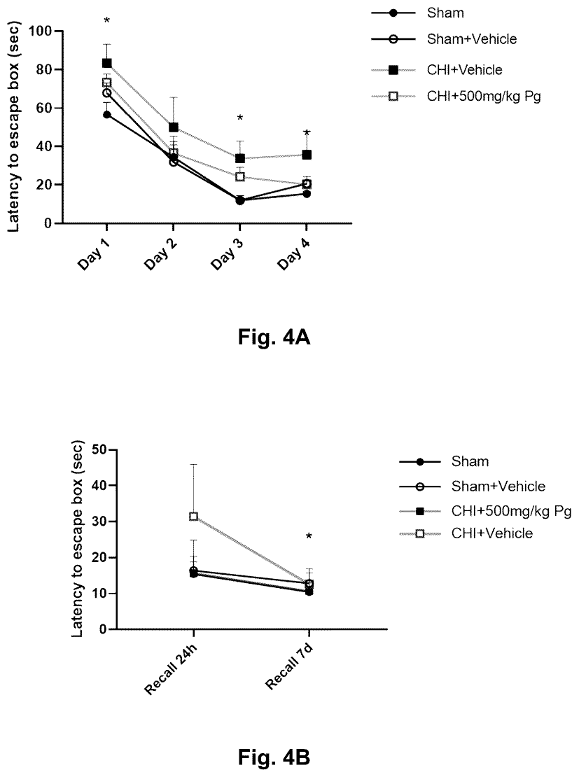

In the Barnes maze (BM) test that was performed two weeks after the trauma (3 days after the last treatment with the Pg essential oil) a significant difference was found between the treated and non-treated group of traumatized animals, whereby the learning curve of the treated mice was similar to the learning curve of the control, uninjured group (sham, and sham+vehicle), and had a faster learning rate compared to non-treated mice (FIG. 3A) the results summarized in FIG. 3A represent 4 repeated experiments where animals treated with 500 mg/kg Pg essential oil were treated with 2 different batches/extractions. In addition, significant differences were found in recall tests 7 days after learning (FIG. 3B). A significant improvement in the learning curves was found in the lower dose of 200 mg/kg in the 24 hours recall test, however this dose did not affect the memory 7 days after learning. No significant differences were found between CHI treated with the high dose between 24 h and 7 days following acquisition indicating this treated group had a long-term memory as the Sham groups (FIG. 3B). The improvement that was seen immediately after treatment with the Pg essential oil was maintained also two months after the trauma (more than one month after completion of the treatment) wherein the learning capability of the treated mice was maintained, and they presented better results than untreated mice (FIGS. 4A and 4B).

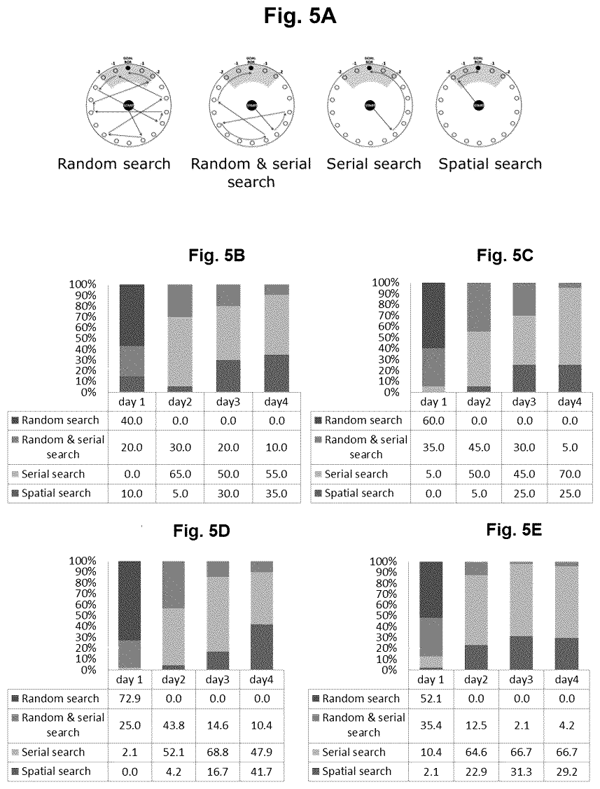

In addition, when the learning strategy of the mice was examined, it was evident that the learning in mice with a head trauma that were treated with the Pg essential oil (500 mg/kg) was based on memory and not on random search, as seen in the learning strategies of Sham animals (FIG. 5).

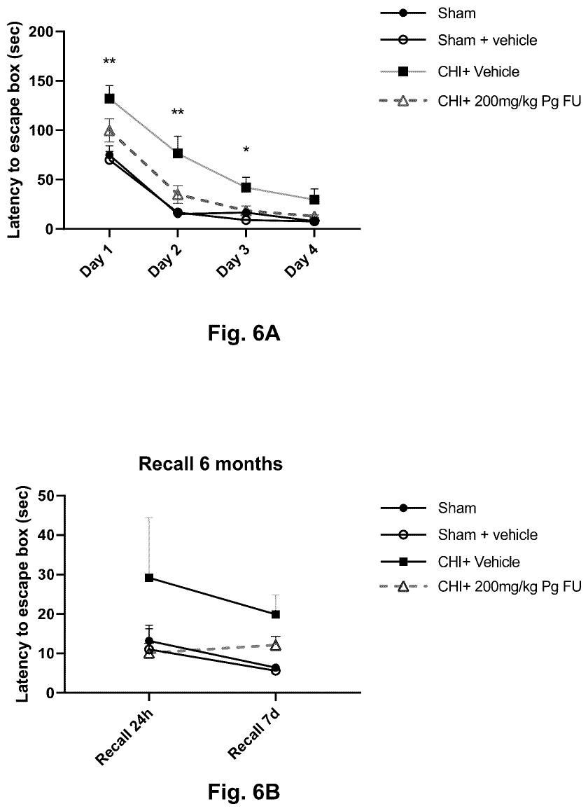

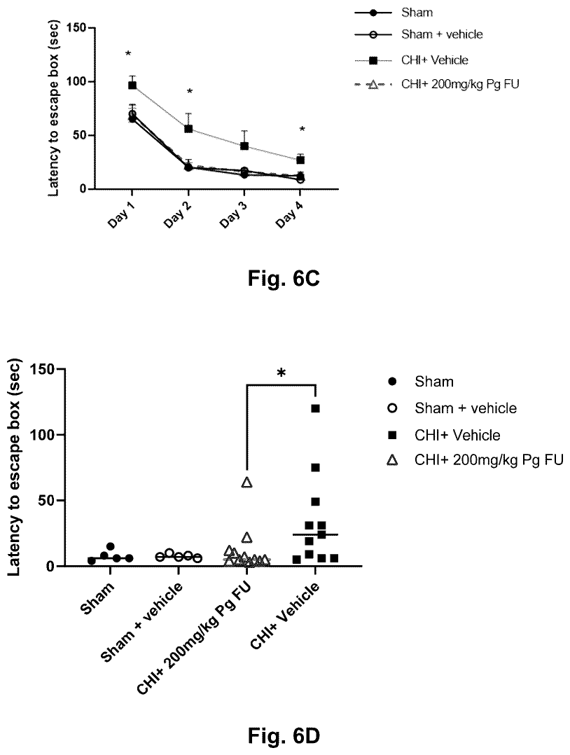

FIG. 6A shows results of mice tested in the BM six months after the trauma. Briefly, mice were subjected to CHI and treated with 200 mg/Kg Pg essential oil for 2 weeks post injury plus one dose once a week as a follow up (FU) treatment. As can be seen in FIG. 6A mice that received initial treatment and a follow up performed significantly better than the CHI+vehicle group.

FIG. 6B shows results of mice undergoing BM recall test (Recall 6 months from the trauma, and 5.5 months after the last treatment with Pg essential oil) performed 24 hours (24 h) and 7 days (7 d) after completion of learning. As can be seen in FIG. 6B mice that received initial treatment with 200 mg/Kg Pg essential oil for 2 weeks post injury plus one dose once a week as a follow up (FU) treatment performed significantly better than the CHI+vehicle group, in both time points, namely 24 hours and 7 days after completion of learning.

FIG. 6C shows results of mice tested in the BM 12 months (one year) after the trauma. Briefly, mice were subjected to CHI and treated with 200 mg/Kg Pg essential oil for 2 weeks post injury plus one dose once a week as a follow up (FU) treatment. As can be seen in FIG. 6C mice that received initial treatment and a follow up performed significantly better than the CHI+vehicle group. FIG. 6D shows results of a BM recall test performed with this group of mice 12 months after the trauma. As can be seen in FIG. 6D mice that received initial treatment and a follow up had a significantly better memory performance as compared with the CHI+vehicle group. The 1 year results show that the treatment with the Pg essential oil can be carried out for a long period of time and its effects are maintained and remain significant. No toxic effects or mortality due to chronic use (1 year) were observed.

Another group of mice received the initial treatment session and a second treatment session 9 months after the CHI. Briefly, mice were subjected to CHI and treated with 500 mg/Kg Pg oil for 2 weeks post injury. Nine months after the trauma, the mice received an additional treatment of 500 mg/Kg Pg for two weeks.

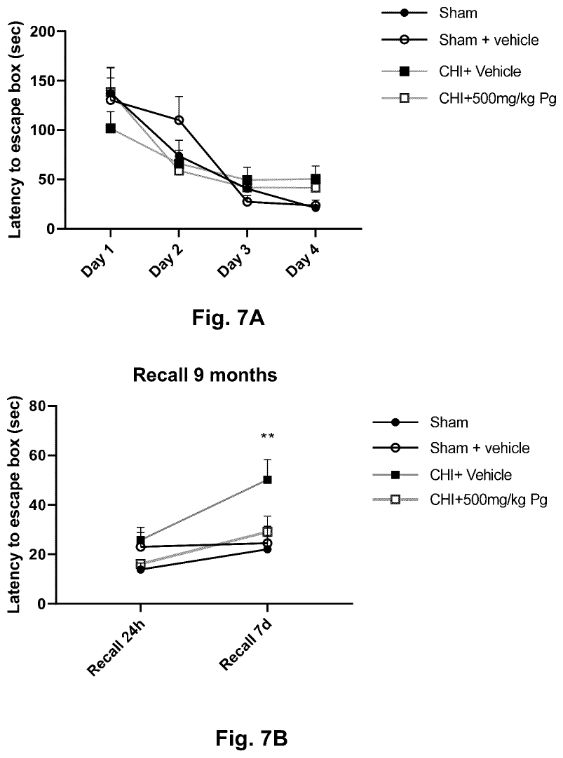

FIG. 7A shows results of these mice tested in the BM nine months after the trauma (after receiving the second treatment session). As can be seen in FIG. 7A there is no significant effect of the treatment on the learning capability.

FIG. 7B shows results of mice undergoing BM recall test (Recall 9 months from the trauma, and 8.5 months after the last treatment with Pg oil, immediately after receiving a second treatment session) performed 24 hours (24 h) and 7 days (7 d) after completion of learning. As can be seen in FIG. 7B the treated mice performed significantly better than the CHI+vehicle group. The results are especially significant 7 days after completion of learning.

These results suggest an impressive effect on memory capabilities.

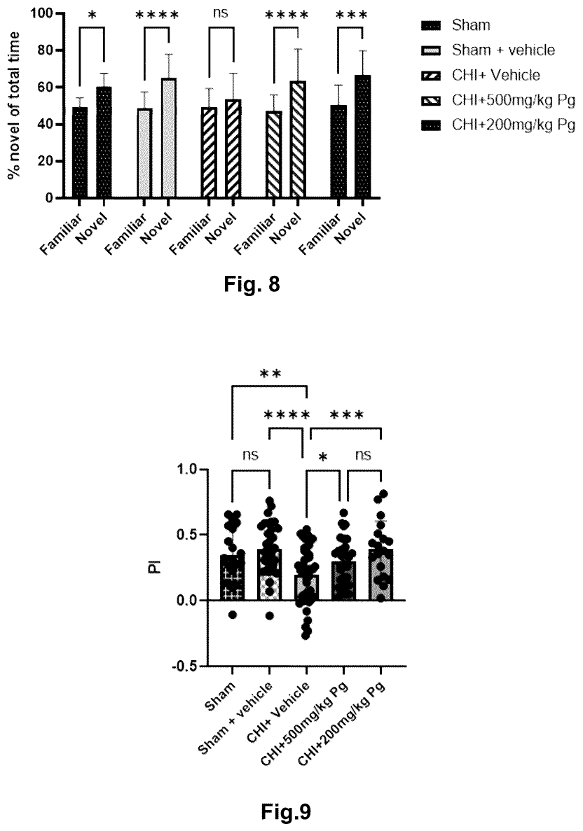

In a Novel Object Recognition (NOR) test performed 23 days after trauma, a significant improvement was found in the memory capability of the treated mice, whereby 4 hours after the familiar object was replaced with a novel object, the percent of the time that the treated mice spent by the novel object was significantly higher than that spent by untreated mice which appeared not to have any memory of the familiar object. This effect was observed in both tested doses 200 mg/kg, and 500 mg/kg (FIG. 8). These effects were not noticed 7 or 9 months after the injury following 2 weeks of additional treatment.

Mean Preference index (PI) in a Y maze test performed one month after the trauma, and 18 days after the last treatment with Pg essential oil.

In a spatial memory test (Y maze test) performed one month after the trauma (two weeks after completion of treatment) it was shown that mice treated with the 500 mg/kg dose performed significantly better than untreated mice in the Y maze test. Mice who received the lower dose (200 mg/kg) also performed better than untreated mice, however in a less significant manner (FIG. 9).

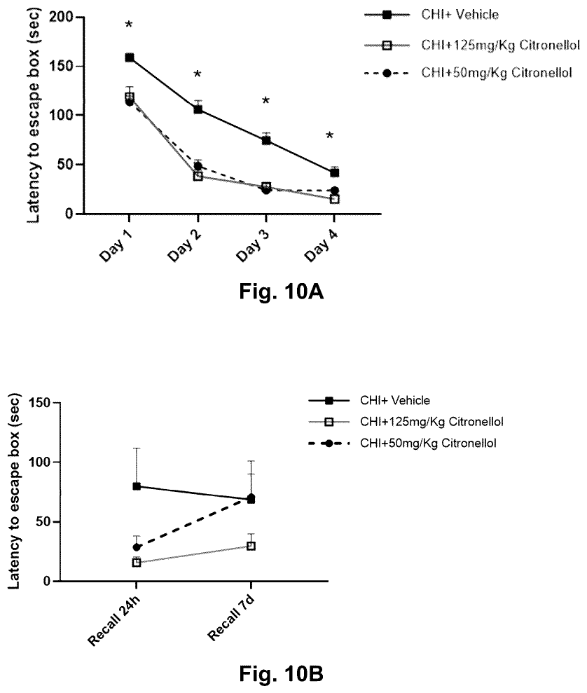

In order to evaluate the effect of citronellol, mice were subjected to a Barnes maze (BM) acquisition test performed 2 weeks after the trauma, and 3 days after the last treatment with Citronellol (125 mg/kg or 50 mg/kg). A significant improvement in the learning curve of treated animals was found in both doses (FIG. 10A) and a significant effect in the long-term memory test (a BM recall test (Recall 2 weeks) performed 24 hours (24 h) and 7 days (7 d) after completion of learning) was found only at the high dose (FIG. 10B).

### Example 3

Testing the Efficacy of Treatment with the Pg Essential Oil—Biochemical Parameters

Upon completion of the behavioral experiments the mice were sacrificed, and their brains underwent histological and biochemical analyses to examine various characteristics of inflammation that appear at the primary and secondary stages of brain damage.

Histological Analysis: Lesion Area. Lateral Ventricle Size and Hippocampal Cell Count:

At 30 d after injury, sham, treated and vehicle control mice underwent deep anesthesia, and perfusion with ice-cold saline. The brains were removed rapidly and frozen at −80° C. and sectioned to 10 μm coronal slices 200 μm apart between bregma+1.78 mm and bregma −2.54 mm. Regions representing the damage area and remote areas were collected. Sections were stained with hematoxylin-eosin (H&E). Briefly, slices were fixed with 4% PFA for 10 min, washed and stained with hematoxylin (Sigma-Aldrich, St. Louis, MO, USA) for about 5 min, rinsed in tap water and then in 0.3% acid alcohol until the background became colorless, washed in tap water, stained with eosin (Sigma-Aldrich, St. Louis, MO, USA) for about 2 min, washed, mounted, and covered. Images of the entire hemisphere were captured with a 0.5 mm light microscope lens for lesion and ventricular measurements and a ×200 lens for hippocampal area and cell count. Regions of interest (ROIs) were measured with ImageJ software (National Institutes of Health, Bethesda, MD, USA).

The area of the lateral ventricles was measured 30 d post CHI. The percentage of damaged tissue (lesion area) 30 d post injury was measured by rotating and placing the contralateral hemisphere image behind that of the ipsilateral one and tracing and measuring the upper-left quarter of the slice (the ipsilateral cortex) with ImageJ software (National Institutes of Health, Bethesda, MD, USA). Lesion area was calculated by dividing the size of the injured area by the area of the whole brain.

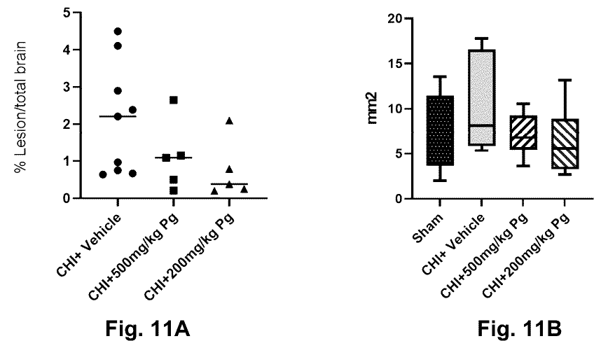

FIGS. 11A and 11B show the results of a quantification of the lesion area and the left ventricle volume, respectively. No significant differences were found between the groups using One Way ANOVA.

The hippocampal cells were counted 30 d post injury by using higher magnification microscope images (×200) for each hippocampus (ipsilateral and contralateral). Three images of different regions were captured from 4-5 different sections/mice: CA1, CA3 and DG (dentate gyrus). The ROI (namely the nuclei layer of the CA1, CA3 and DG,) was marked and the area was measured with ImageJ software (NIH). To count the number of neurons in the hippocampus, ImageJ software (NIH) was used to first filter nuclei larger than 20 μm² and exclude the glial nuclei, which are significantly smaller, and then the neuronal nuclei in the ROI were counted. TBI+200 mg/Kg Pg oil n=6, TBI+500 mg/Kg Pg oil n=7, TBI+vehicle n=8, Sham+vehicle n=6 and Sham n=6.

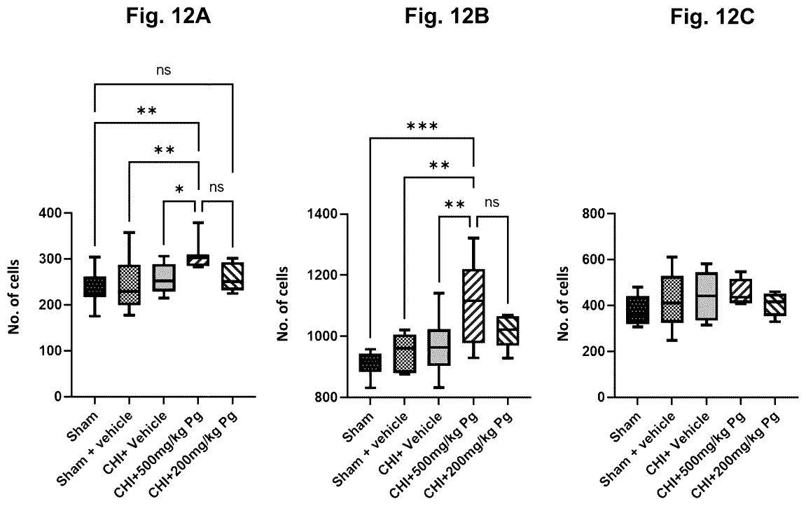

FIG. 12A-C show neuronal cell count in various regions of the left hemisphere hippocampus: CA1 (FIG. 12A), DG (dentate gyrus) (FIG. 12B), and CA3 (FIG. 12C).

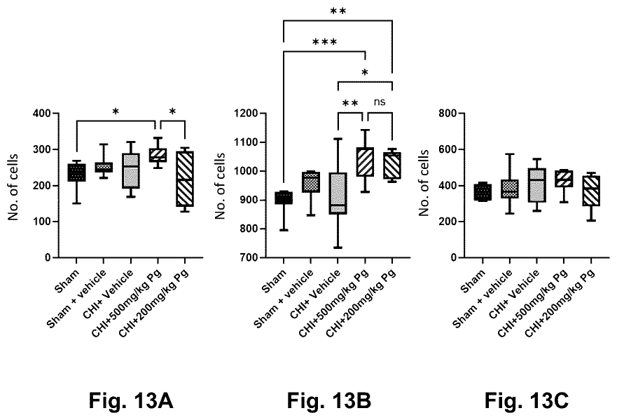

FIG. 13A-C show neuronal cell count in various regions of the right hemisphere hippocampus: CA1 (FIG. 13A), DG (dentate gyrus) (FIG. 13B), and CA3 (FIG. 13C).

Apparently, the treatment causes in increase in the number of neurons in brain areas that are relevant to cognitive functions. Without wishing to be bound by theory, the higher cell numbers may infer a higher cell survival rate due to a more protective environment, and/or proliferation of cells originating from stem cells. Interestingly, cell numbers were higher both in the lesioned hemisphere and in the second hemisphere that was not directly affected by the trauma, indicating that not only the primary damage may be ameliorated but also secondary damage resulting for example from inflammation. Since the Pg treatment is administered systemically both the lesioned area as well as remote regions are affected by the treatment.

### Immunohistochemistry

At 30 d post CHI, brain slices were also stained for immunohistochemical evaluation using various cell markers. Briefly, slices were fixed with 4% PFA for 10 min, washed with PBST and blocked with 10% normal donkey serum (NDS, Abcam, Cambridge, United Kingdom) for 1 h, washed and exposed to antibodies that included glial fibrillary acidic protein (GFAP, 1:1000; Dako, Glostrup, Denmark) an astrocyte cell marker, CREB (1:1000, Abcam, Cambridge, United Kingdom), neurofilament (NF) (anti-neurofilament 1:1000 Sigma-Aldrich, St. Louis, MO, USA), Ionized Calcium Binding Adapter Molecule 1 (Iba-1, 1:500, Abcam, Cambridge, United Kingdom), CD206 (1:100, Santa Cruz, CA, USA) in 2% NDS for 45 min. Dylight 488 (1:300, Abcam) and Cy3 (1:1000, Jackson ImmunoResearch, West Grove, PA, USA) served as secondary antibodies. To avoid positive artifacts, the Hoechst stain was used to visualize the nuclei. An average of 3 images/brain region/side/mouse were captured under a fluorescence microscope, with the same exposure time for each antibody. The mean fluorescence values were measured with ImageJ software (NIH) in specific ROI. For the GFAP staining—TBI+200 mg/Kg Pg oil n=4, TBI+500 mg/Kg Pg oil n=7, TBI+vehicle n=7, Sham+vehicle n=3 and Sham n=4. For the NeuN and NF staining—TBI+500 mg/Kg Pg oil n=5, TBI+vehicle n=3, Sham+vehicle n=1 and Sham n=2.

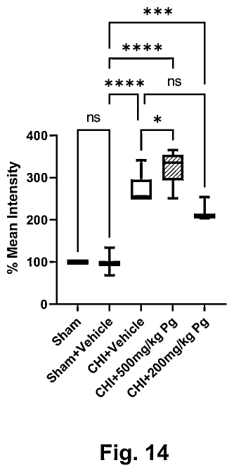

FIG. 14 shows astrocyte staining in the ipsilateral cortex, using the astrocyte marker GFAP. As can be seen in the figure there is an elevation in GFAP staining following CHI in both Pg treated and vehicle treated mice in the cortex adjacent to injury.

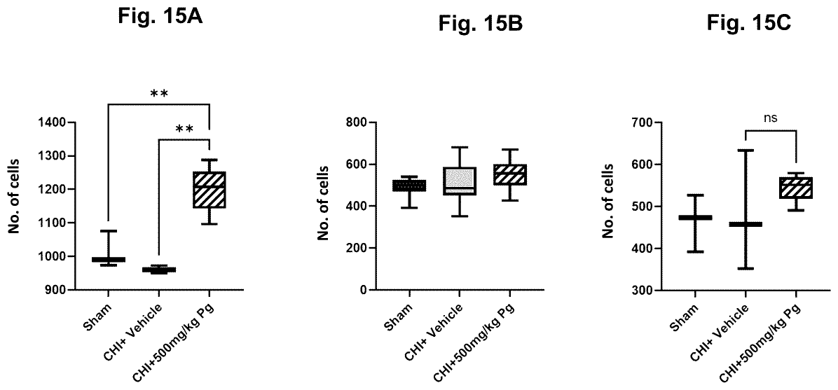

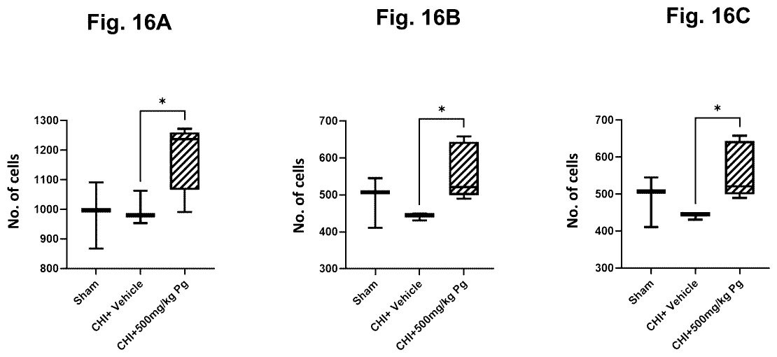

NeuN staining, which identifies neuronal cell bodies and thus allows the direct counting of neuronal cells, is shown in FIG. 15 for the ipsilateral cortex, and in FIG. 16 for the contralateral cortex.

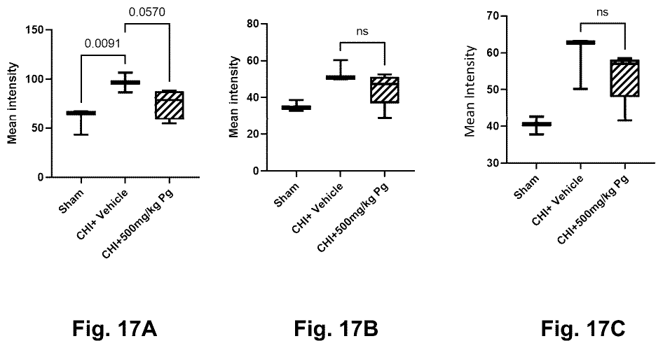

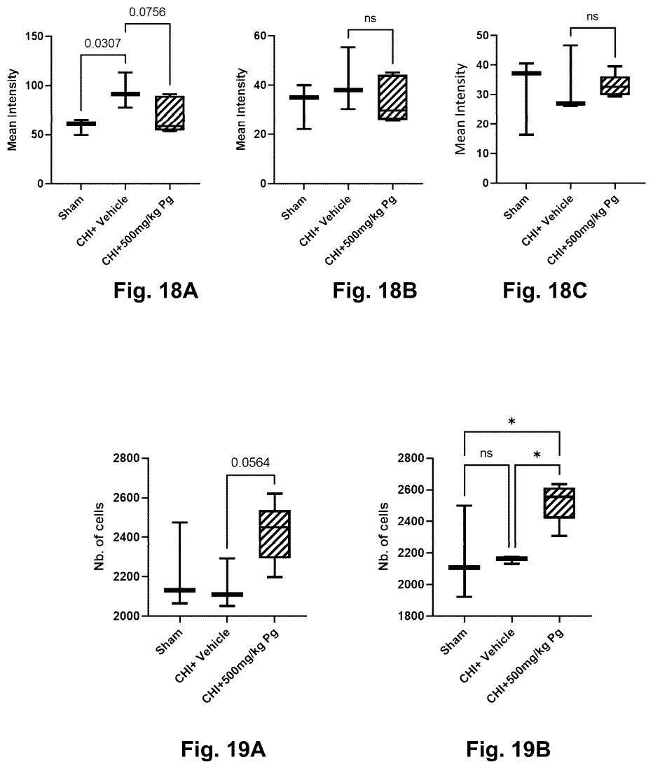

NF200 (neurofilament) staining, which identifies neuronal axons, is shown in FIG. 17 for the ipsilateral cortex, and in FIG. 18 for the contralateral cortex.

The transcription factor CREB is expressed mainly in neurons and has a well-documented role in neuronal plasticity and long-term memory formation in the brain. It has been shown to be integral in the formation of spatial memory. CREB staining is shown in FIG. 19A for the ipsilateral cortex, and in FIG. 19B for the contralateral cortex.

Without wishing to be bound by theory, these histological results show a beneficial effect of the Pg treatment which may serve as the basis for the improved cognitive performance found in animals treated with Pg following TBI.

### Confocal Microscopy and Quantification of Microglia's Morphology

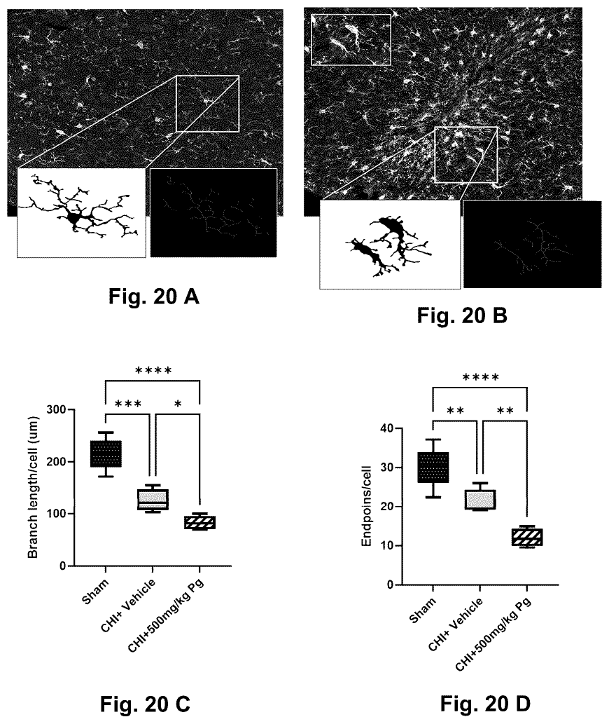

Images were acquired using TCS SP8 Leica Confocal microscope (Leica, Germany). The same settings were applied to all images within the same experiment. All analyses were carried out using open-source ImageJ/Fiji software. For microglia morphology analyses, Iba-1 positive cells were used. Single microglia cells were selected in areas where a single cell could be identified. After the background was subtracted and a threshold was applied, images were converted to binary and then skeletonized (FIG. 20A shows a representative microglial cell in a sham operated mouse; FIG. 20B shows representative microglial cells in an injured mouse). The functions Fractal Analysis FracLac plugin and Summarize skeleton in ImageJ/Fiji software were used to quantify the average branch length and the average number of endpoints in each cell (as described in Young and Morrison, 2018). The values of each individual cell were used to obtain the group mean and standard deviation for each condition and experiment. A macro was developed in ImageJ/Fiji software to automate the analysis.

FIG. 20C shows the branch length and FIG. 20D shows the number of endpoints in sham operated mice, injured mice (CHI+vehicle) and injured mice treated with 500 mg/kg Pg essential oil. The treatment reduces the branch length and the number of endpoints, indicating a shift of the inflammatory response into a “protective” type of microglia namely, M2 microglia. M1 microglia induce inflammation and neurotoxicity, while M2 microglia induce anti-inflammatory responses and neuroprotection. These results are further supported by the CD206 positive/Iba-1 positive staining (see next paragraph). CD206 is widely recognized as a representative M2 microglial marker.

### Cell Count of Microglia Double Positive to CD206+ and Iba1+:

The same settings were applied to all images. To identify the phenotypes of microglia cells in the ipsilateral cortex after the trauma, double staining of CD206 (M2 marker) with Iba1 was performed. CD206+ cells that are also Iba1+ were counted manually with ImageJ software (NIH), divided by the total number of Iba1+ cells and converted to percentage.

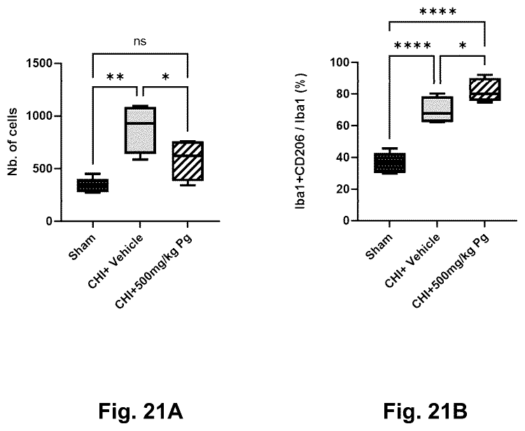

FIG. 21A shows the total no. of Iba-1+ cells in the lesioned area of the left cortex and FIG. 21B shows the percent of double stained cells (Iba-1+ and CD206+) in sham operated mice, injured mice (CHI+vehicle) and injured mice treated with 500 mg/kg Pg essential oil. The analysis was performed on brain sections taken 30 days after CHI.

As can be seen in FIG. 21A there is an elevation of the inflammatory response, as expressed by the number of microglia, around the lesion area, as expected. Pg treatment significantly reduced the number of microglia in the lesioned cortex. Moreover, the numbers of double positive cells (iba-1positive CD206 positive) were significantly elevated in the treated group indicating an elevation in M2 microglia cells Thus, taken together these results indicate that the Pg treatment immunomodulates the inflammatory response towards a protective profile rather than towards harmful and neurotoxic effects.

### Microglia Phagocytosis:

The effect of the Pg oil on microglial phagocytosis was tested in vitro. Real-time live-cell quantification of the phagocytosis was performed using pHrodo Bioparticles. The pHrodo-based system exploits the acidic environment of the phagosome to quantify phagocytosis, as pHrodo Bioparticles are engulfed by microglia cells and enter the acidic phagosome, a substantial increase in fluorescence is observed. BV2 cells (10,000 cells per well) were seeded 24 h prior to the experiment in 1% FBS in DMEM. For the experiment medium was switched to EMEM and 10 μg pHrodo red E. coli bioparticles (Sartorius) were loaded into each well in a 96 well plate and treated or not with 20 μg/ml Pg oil that was first diluted in DMSO. Pictures were taken by Incucyte apparatus (Sartorius), objective ×10, every 30 min.

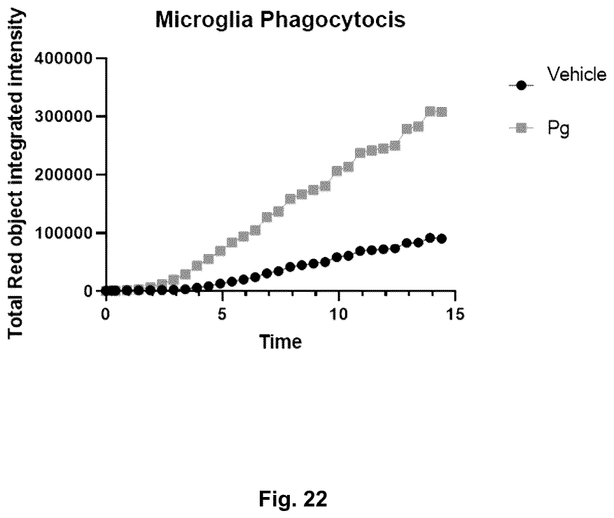

FIG. 22 shows a time-course of phagocytosis of the pHrodo red E. coli bioparticles by the BV2 mouse microglial cells. The Pg oil had a dramatic effect on the phagocytic activity of the microglia.

These results further point to the neuroprotective and anti-inflammatory effect of Pg using different assay.

### Statistical Analysis:

For the statistical analyses, a commercially available computer software (StatView Software) or GraphPad Prism 9 (San Diego, California) was used. The treatments were the independent variables, and the outcomes of the CHI parameters were the dependent variables. Significance was tested by using one or two-way analysis of variance, followed by Fisher's PLSD post-test method. The repeated measures ANOVA test was used to evaluate the group main effect in NSS, and BM followed by t-test to compare each group versus the vehicle control per day.

### Biochemical Analysis of Hippocampus and Cortex Homogenates

For the biochemical analysis the brains were separated to different brain regions: cortex and hippocampus, right and left (ipsilateral and contralateral to injury) and were frozen immediately in liquid nitrogen.

Preparation of cortex and hippocampus homogenates for measuring cytokine and pCREB levels: Samples of the different brain areas were homogenized separately in a cold lysis buffer (Cell Signaling) containing [20 mM Tris-HCl (pH 7.5), 150 mM NaCl, 1 mM Na₂EDTA, 1 mM EGTA, 1% Triton, 2.5 mM sodium pyrophosphate, 1 mM beta-glycerophosphate, 1 mM Na₃VO₄, 1 μg/ml leupeptin], or in a lysis buffer from an EnzChek Caspase-3 kit (E-13183) prepared according to Manufacturer's instructions comprising 10 mM TRIS, 100 mM NaCl, 1 mM EDTA, 0.001% Triton X-500) together with EZBlock™ Protease Inhibitor Cocktail, EDTA-Free, (Biovision).

The cortex samples underwent lysis at a ratio of 0.065 gr tissue/ml and the hippocampus samples underwent lysis at a ratio of 0.054 gr tissue/ml. The homogenization was performed using Micro-Tube homogenizer. The homogenates were frozen in liquid nitrogen and thawed three times and then centrifuged (10,000 g for 10 minutes at 4° C.), the supernatant was collected, aliquoted and frozen at −80° C. Protein levels in the homogenates were measured by Bradford analysis.

The levels of TNF-α, IL-6, IL-1β and CREB in the brain homogenates were measured using commercial ELISA kits according to Manufacturer's instructions (IL-6 and TNFα—Diaclone Research, France; IL-1β—R&D Systems, Canada; Pathscan phospo-CREB and total CREB—Cell signaling, MA, USA).

Preparation of cortex and hippocampus homogenates for enzymatic measurements: Cortex and hippocampus tissues were homogenized in a lysis buffer from an EnzChek Caspase-3 kit prepared according to Manufacturer's instructions 20× buffer=200 mM TRIS, pH7.5, 2M NaCl, 20 mM EDTA, 0.2% Triton X-100). The cortex samples underwent lysis at an average ratio of 0.074 gr tissue/ml and the hippocampus samples underwent lysis at a ratio of 0.04 gr tissue/ml. The homogenization was performed in Eppendorf tubes using Micro-Tube homogenizer. The homogenates were frozen in liquid nitrogen and thawed three times and then centrifuged (10,000 g for 10 minutes at 4° C.), the supernatant was collected, aliquoted and frozen at −80° C. Protein levels in the homogenates were measured by Bradford analysis.

The enzymatic activity of acetylcholine esterase was tested using a commercial kit (K764, Biovision, CA, USA).

Preparation of cortex and hippocampus homogenates for measuring lipid peroxidation: Cortex and hippocampus tissues were homogenized at ta ratio of 0.06 gr tissue/ml and 0.031 gr tissue/ml, respectively, in a Radioimmunoprecipitation assay (RIPA) buffer (Cayman chemical, 10010263) prepared according to Manufacturer's instructions and tittered using hydrogen chloride to pH=7.6. The homogenization was performed in Eppendorf tubes using Micro-Tube homogenizer. The homogenates were frozen in liquid nitrogen and thawed three times and then centrifuged (1,600 g for 10 minutes at 4° C.), the supernatant was collected, aliquoted and frozen at −80° C. Protein levels in the homogenates were measured by Bradford analysis.

Bradford analysis: The protein concentration in the various brain areas and the cell lysates was measured using the Bradford method. BSA dissolved in DDW (double distilled water) to a concentration of 1.4 mg/ml was used as a standard for the calibration curve. A volume of 20-30 μl from each sample was transferred to an Eppendorf tube, DDW was added to a total volume of 800 μl and 200 μl of the Bradford reagent X5 were added. The diluted samples were vortexed and then incubated in the dark for 20 minutes at room temperature. At the end of the incubation the tubes were vortexed once again and 200 μl were transferred in duplicate to a 96 well-plate for reading in a plate reader at a wavelength of 595 nm to determine protein content.

Measuring lipid peroxidation: Lipid peroxidation was measured using the TBARS (Thiobarbituric acid reactive substances) kit (Cayman Chemicals) according to Manufacturer's instructions. Compounds that react with Thiobarbituric acid (TBA) are byproducts of lipid peroxidation and they can be identified by the TBARS method. This test is based on measuring Malondialdehyde (MDA) which is a common product of lipid peroxidation.

### Results

Only the left hemisphere was analyzed as this is the area that underwent the trauma.

### TNFα Levels

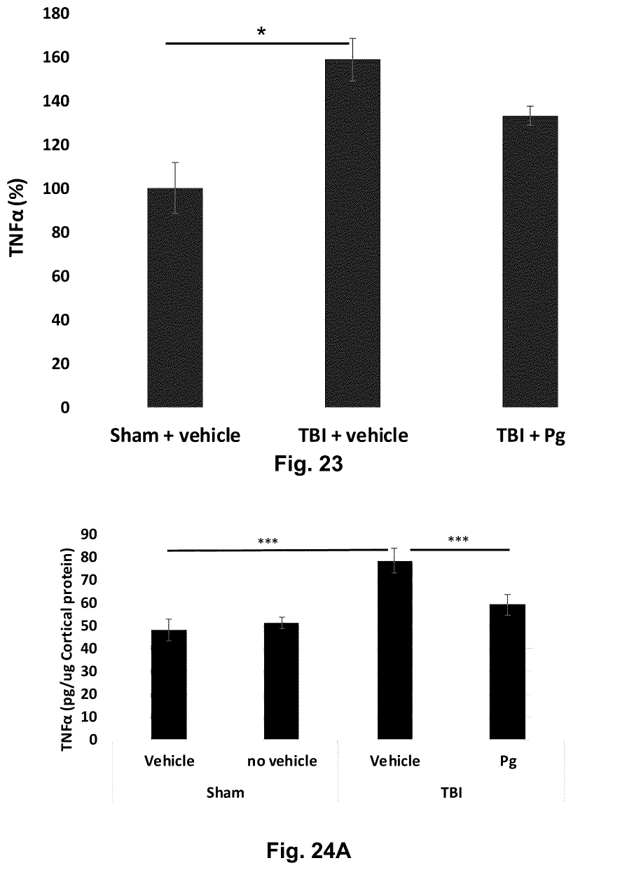

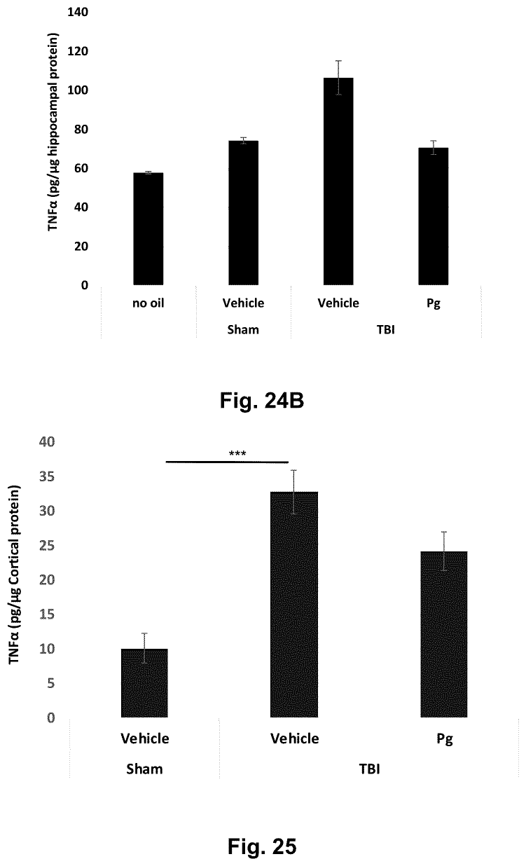

As shown in FIG. 23, 24 hours after trauma the levels of TNFα which is a proinflammatory cytokine were increased by 1.6 in the lesioned cortex. Treatment with the Pg oil inhibited this increase by about 50%. A significant increase in the levels of TNFα was also observed in the cortex and hippocampus of the mice 11 days after trauma as compared with control un traumatized mice (FIG. 24). Treatment with the Pg oil prevented almost completely the trauma-induced increase in TNFα.

A significant increase in the levels of TNFα was also observed in the cortex of mice 30 days after trauma (FIG. 25). Treatment with Pg oil partially inhibited the trauma-induced increase in TNFα.

### IL-1β Levels

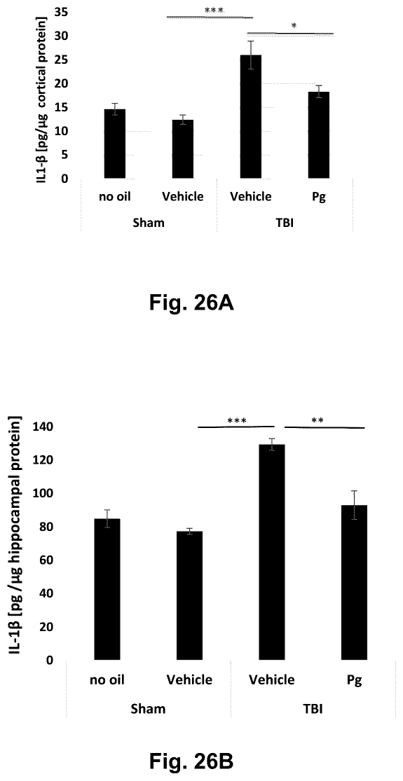

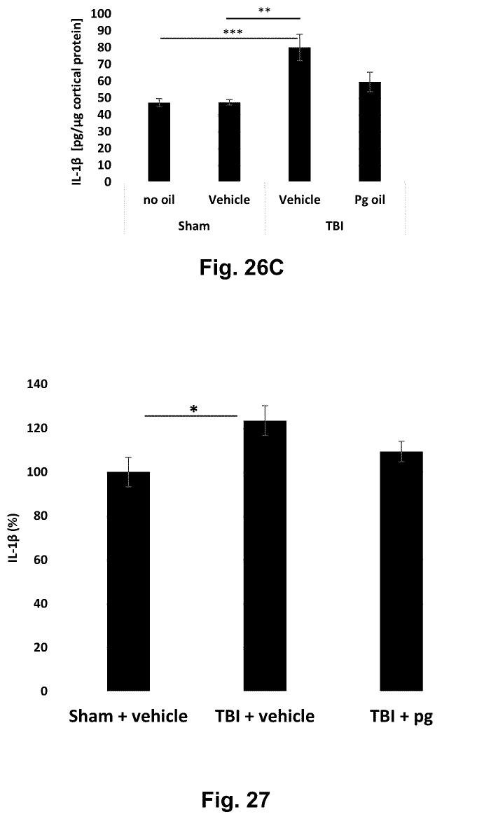

Feeding with the Pg oil reduces the levels of IL-1β in the cortex 11 days, and 30 days after trauma (FIG. 26A, C).

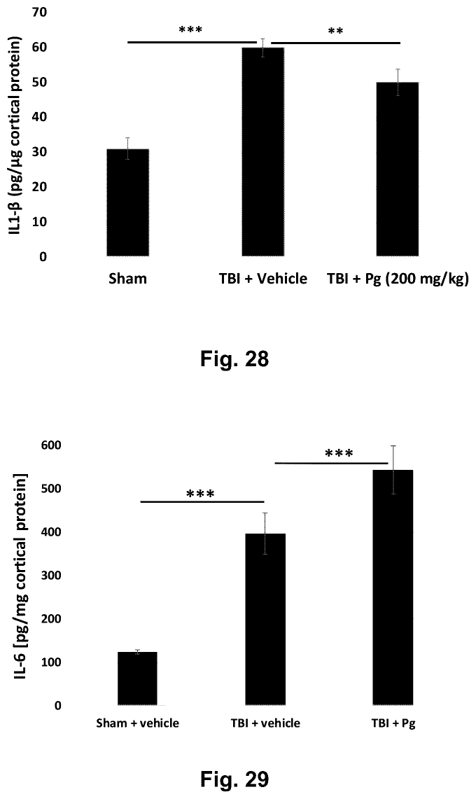

In addition, as can be seen in FIGS. 26B and 27, similar results were obtained in the hippocampus 48 hours or 11 days after trauma. As shown in FIG. 28, feeding the mice with the lower dose of 200 mg/kg also resulted in reduction in the levels of IL-10 in the cortex, 30 days after the trauma.

### IL-6 Levels

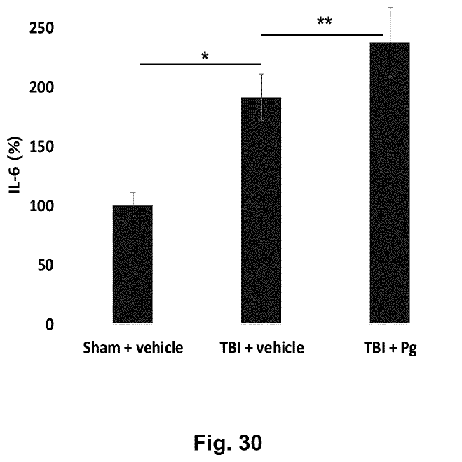

Feeding with the Pg oil before the trauma increased the levels of IL-6 in the cortex 24 hours, and 48 hours after trauma (FIGS. 29 and 30).

### Activity of the Enzyme Acetylcholine Esterase in the Cortex

Head injury causes neuronal cell death. Neuronal cell death is reflected, among others, in reduced enzymatic activity of acetylcholine esterase. As can be seen in FIG. 31, the activity of the enzyme in the cortex of an animal that suffered a head trauma is lower than its activity in a control uninjured animal. Feeding with the Pg essential oil prevented about 50% of this reduction.

### Lipid Peroxidation in the Cortex

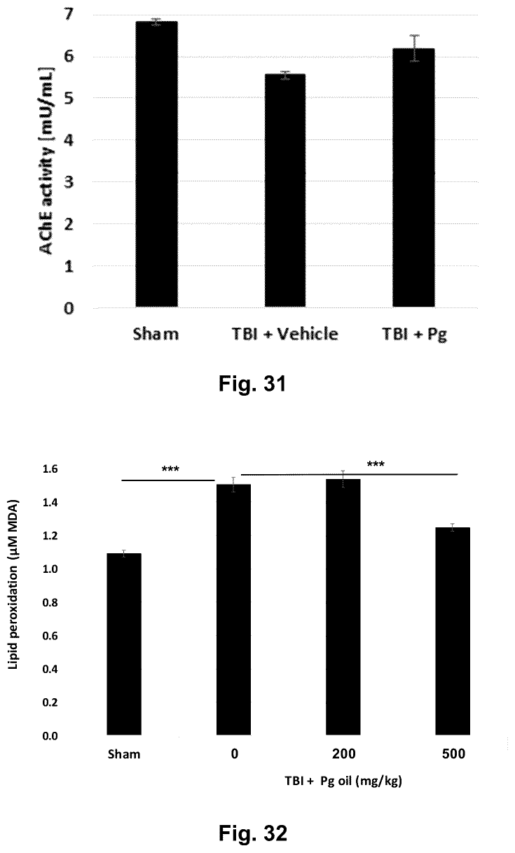

As can be seen in FIG. 32, a head trauma causes an increased lipid peroxidation. Feeding with the Pg oil (500 mg/kg) prevented about 61% of the trauma-induced increased lipid peroxidation.

pCREB Levels in the Cortex and Hippocampus

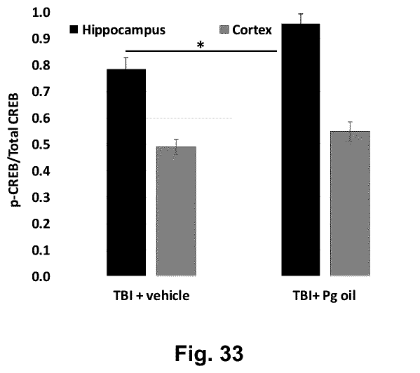

As can be seen in FIG. 33, feeding with the Pg oil causes a significant increase in the levels of pCREB.

### Example 4

Administering Pg Essential Oil to Patients Suffering from TBI

Patients suffering from traumatic brain injury are treated with the Pg oil of the invention.

The treatment is by oral or nasal administration and is given immediately after the trauma followed by a 12-days treatment session in which the subject receives a total of 9 treatments, administered once a day or once in two or three days. The following dosages are administered: 5 mg/kg (for intranasal administration), and 16 mg/kg or 40 mg/kg (for oral administration). Optionally, an additional treatment of 40 mg/kg is given for two weeks, 9 months after the injury.

The patients undergo cognitive evaluation to assess efficacy of treatment.

1. A pharmaceutical or nutritional composition for treating, preventing, or ameliorating traumatic brain injury in a subject and for improving cognitive function of a subject suffering from traumatic brain injury, the composition comprising essential oil extracted from Pelargonium graveolens and one or more physiologically acceptable carriers.

2.-10. (canceled)

11. The composition according to claim 1 wherein said composition is administered at a concentration of between about 1 mg/kg and 50 mg/kg.

12. The composition according to claim 1 wherein said composition is administered once, twice, three times or more daily, or once every two days or every three days for a treatment session of between about 5 days and four weeks, or more following the TBI, or administered in a prophylactic manner to subjects at risk of TBI.

13. The composition according to claim 1 wherein said composition is administered in a treatment session that comprises between about 8 and about 12 daily treatments, e.g., 9 daily treatments per session.

14. The composition according to claim 13, wherein said composition is administered in a treatment session that comprises between about 8 and about 12 daily treatments, e.g., 9 daily treatments per session, followed by chronic administration once a week.

15. The composition according to claim 1 wherein at least one additional treatment session with said composition is administered to said subject between about 4 months, 5 months, 6 months, 7 months, 8 months, 9 months, 10 months, 11 months, 12 months, or more following the TBI.

16. The composition according to claim 1 wherein said composition is provided orally at a concentration of between about 5 mg/kg and about 50 mg/kg once a day or once every two or three days for a treatment session that comprises between about 8 and about 12 daily treatments per session, e.g., 9 daily treatments per session.

17. The composition according to claim 1 wherein said composition is provided by intranasal administration at a concentration of between about 1 mg/kg and about 5 mg/kg once a day or once every two or three days for a treatment session that comprises between about 8 and about 12 daily treatments per session, e.g., 9 daily treatments per session.

18. The composition according to claim 1 wherein said composition is provided by inhalation at a concentration of between about 0.1% and about 10% once a day or once every two or three days for a treatment session that comprises between about 8 and about 12 daily treatments per session, e.g., 9 daily treatments per session.

19.-22. (canceled)

23. A method of treating, preventing, or ameliorating TBI, or of improving cognitive function of a subject suffering from traumatic brain injury the method comprising administering to a subject in need thereof a therapeutically effective amount of Pelargonium graveolens essential oil extract or a composition comprising Pelargonium graveolens essential oil extract.

24.-32. (canceled)

33. The method according to claim 2, wherein said composition is administered at a concentration of between about 1 mg/kg and 50 mg/kg.

34. The method according to claim 2, wherein said composition is administered once, twice, three times or more daily, or once every two days or every three days for a treatment session of between about 5 days and four weeks, or more following the TBI, or administered in a prophylactic manner to subjects at risk of TBI.

35. The method according to claim 2, wherein said composition is administered in a treatment session that comprises between about 8 and about 12 daily treatments, e.g., 9 daily treatments per session.

36. The method according to claim 35, wherein said composition is administered in a treatment session that comprises between about 8 and about 12 daily treatments, e.g., 9 daily treatments per session, followed by chronic administration once a week.

37. The method according to claim 2, wherein at least one additional treatment session with said composition is administered to said subject between about 4 months, 5 months, 6 months, 7 months, 8 months, 9 months, 10 months, 11 months, 12 months, or more following the TBI.

38. The method according to claim 2, wherein said composition is provided orally at a concentration of between about 5 mg/kg and about 50 mg/kg once a day or once every two or three days for a treatment session that comprises between about 8 and about 12 daily treatments per session, e.g., 9 daily treatments per session.

39. The method according to claim 2, wherein said composition is provided by intranasal administration at a concentration of between about 1 mg/kg and about 5 mg/kg once a day or once every two or three days for a treatment session that comprises between about 8 and about 12 daily treatments per session, e.g., 9 daily treatments per session.

40. The method according to claim 2, wherein said composition is provided by inhalation at a concentration of between about 0.1% and about 10% once a day or once every two or three days for a treatment session that comprises between about 8 and about 12 daily treatments per session, e.g., 9 daily treatments per session.

41.-46. (canceled)

47. A method of treating, preventing, or ameliorating TBI, or for improving cognitive function of a subject suffering from traumatic brain injury, the method comprising administering to a subject in need thereof a therapeutically effective amount of isolated citronellol.

48. (canceled)

49. The method of claim 47, wherein said isolated citronellol is administered at a concentration of between about 1 mg/kg and 15 mg/kg, e.g., 4 mg/kg or 10 mg/kg.
